(19) United States
(12) Patent Application Publication
(10) Pub. No.: US 2016/0235832 A1
(43) Pub. Date: Aug. 18, 2016

(54) Compositions and the Use of Fibrinogen Binding Motif Presence in EFB and COA for Vaccine Against Staphylococcus Aureus and Drug Delivery

(71) Applicants: THE TEXAS A&M UNIVERSITY SYSTEM, College Station, TX (US); UNIVERSITY MEDICAL CENTER UTRECHT, THE NETHERLANDS, Utrecht (NL)

(72) Inventors: Ya-Ping Ko, Houston, TX (US); Magnus Hook, Houston, TX (US); Suzan HM Rooijakkers, Den Bosch (NL); Mary Beth Browning, Houston, TX (US)

(21) Appl. No.: 15/029,896

(22) PCT Filed: Oct. 15, 2014
(86) PCT No.: PCT/US2014/060772

§ 371 Date: Apr. 15, 2016

Related U.S. Application Data
(60) Provisional application No. 61/891,233, filed on Oct. 15, 2013.

(51) Int. Cl. A61K 39/085; G01N 33/577; G01N 33/68; C07K 16/12; C07K 14/31

(52) U.S. Cl. CPC A61K 39/085; C07K 16/1271; C07K 14/31; G01N 33/6854; G01N 33/577; C07K 2317/56; C07K 2317/76; A61K 2039/55516

(57) ABSTRACT

The present disclosure provides methods and composition including vaccines, monoclonal antibodies, polyclonal antibodies, chimeric molecule of an extracellular fibrinogen binding protein (Efb) and targeted agent delivery pharmaceutical composition comprising at least a portion of a modified N-terminus region, at least a portion of a modified C-terminus region, or both, wherein the modified extracellular fibrinogen binding protein results in inhibiting the fibrinogen binding, C3 binding, or both or administering to a subject a pharmacologically effective amount of a vaccine in a pharmaceutically acceptable excipient, comprising a modified extracellular fibrinogen binding protein comprising at least a portion of a modified N-terminus region, at least a portion of a modified C-terminus region, or both, wherein the modified extracellular fibrinogen binding protein results in not shielding the staphylococcus bacterium from recognition by a phagocytic receptor.

### TECHNICAL FIELD OF THE INVENTION

The present invention relates generally to compositions and methods for preventing and treating human and animal diseases including, but not limited to, pathogens.

### REFERENCE TO A SEQUENCE LISTING

The present application includes a Sequence Listing which has been submitted in ASCII format via EFS-Web and is hereby incorporated by reference in its entirety. Said ASCII copy, created on ______, 2014, is named TAMU:2066WO.txt and is ______ KB in size.

### BACKGROUND ART

Without limiting the scope of the invention, its background is described in connection with compositions and methods of treating infection by pathogens. Pathogens present serious health concerns for all animals, including humans, farm livestock, and household pets. These health threats are exacerbated by the rise of strains that are resistant to antibiotic treatment. Staphylococcus aureus is a leading cause of severe bacterial infections in both hospital and community settings. Due to its increasing resistance to antibiotics, development of additional therapeutic strategies like vaccination is required to control this pathogen. Vaccination attempts against S. aureus have not been successful so far and an important reason may be the pathogen's elaborate repertoire of molecules that dampen the immune response. These evasion molecules not only suppress natural immunity but also hamper the current attempts to create effective vaccines.

### DISCLOSURE OF THE INVENTION

The present invention provides vaccine comprising: (a) a pharmacologically effective amount of a vaccine in a pharmaceutically acceptable excipient, comprising a modified extracellular fibrinogen binding protein comprising at least a portion of a modified N-terminus fibrinogen binding region, at least a portion of a modified C-terminus complement protein binding region, or both, wherein the modified extracellular fibrinogen binding protein results in inhibiting the fibrinogen binding, C3 binding, or both; (b) a pharmacologically effective amount of a vaccine in a pharmaceutically acceptable excipient, comprising a modified extracellular fibrinogen binding protein comprising at least a portion of a modified N-terminus fibrinogen binding region, at least a portion of a modified C-terminus complement protein binding region, or both, wherein the modified extracellular fibrinogen binding protein does not shield the surface-bound complement protein, an antibody or both from recognition by a phagocytic receptor; or (c) a pharmacologically effective amount of a vaccine in a pharmaceutically acceptable excipient, comprising a modified extracellular fibrinogen binding protein comprising at least a portion of a modified N-terminus fibrinogen binding region, at least a portion of a modified C-terminus complement protein binding region, or both, wherein the modified extracellular fibrinogen binding protein does not shield the staphylococcus bacterium from recognition by a phagocytic receptor. The present invention provides a chimeric molecule of an extracellular fibrinogen binding protein (Efb) comprising: a N-terminus fibrinogen binding region that binds a fibrinogen; and a C-terminus complement protein binding region that binds a complement protein, wherein the chimeric molecule can modulate complement activity, modulate antibody binding, modulate recognition by a phagocytic receptor or a combination thereof.

The present invention provides a monoclonal and/or polyclonal antibody or antigen-binding fragment thereof that can specifically bind to a portion of a extracellular fibrinogen binding protein comprising a heavy and light chain variable regions that bind at least a portion of a N-terminus fibrinogen binding region of a extracellular fibrinogen binding protein, at least a portion of a C-terminus complement protein binding region of a extracellular fibrinogen binding protein, or both and results in the inhibition of fibrinogen binding, of complement protein binding, inhibition of the shielding of the staphylococcus bacterium from recognition by a phagocytic receptor or a combination thereof.

The present invention provides a pharmaceutical composition comprising a pharmacologically effective amount of a modified extracellular fibrinogen binding protein in a pharmaceutically acceptable excipient, wherein the modified extracellular fibrinogen binding protein comprises at least a portion of a N-terminus fibrinogen binding region, at least a portion of a C-terminus complement protein binding region, or both, wherein the modified extracellular fibrinogen binding protein results in inhibiting the fibrinogen binding, C3 binding, the surface-bound complement protein, an antibody or combination thereof.

The present invention provides a pharmaceutical composition comprising a monoclonal and/or polyclonal antibody or antigen-binding fragment thereof that can specifically bind to a portion of a extracellular fibrinogen binding protein comprising a heavy and light chain variable regions that bind at least a portion of a N-terminus fibrinogen binding region of a extracellular fibrinogen binding protein, at least a portion of a C-terminus complement protein binding region of a extracellular fibrinogen binding protein, or both and results in the inhibition of fibrinogen binding, of complement protein binding, inhibition of the shielding of the staphylococcus bacterium from recognition by a phagocytic receptor or a combination thereof.

The present invention provides a pharmaceutical composition for use in the treatment of an infection comprising (a) a pharmacologically effective amount of a modified extracellular fibrinogen binding protein in a pharmaceutically acceptable excipient, wherein the modified extracellular fibrinogen binding protein comprises at least a portion of a N-terminus fibrinogen binding region, at least a portion of a C-terminus complement protein binding region, or both, wherein the modified extracellular fibrinogen binding protein results in inhibiting the fibrinogen binding, C3 binding, the surface-bound complement protein, an antibody or combination thereof; or (b) a pharmacologically effective amount of a monoclonal and/or polyclonal antibody or antigen-binding fragment thereof that can specifically bind to a portion of a extracellular fibrinogen binding protein comprising a heavy and light chain variable regions that bind at least a portion of a N-terminus fibrinogen binding region of a extracellular fibrinogen binding protein, at least a portion of a C-terminus complement protein binding region of a extracellular fibrinogen binding protein, or both and results in the inhibition of fibrinogen binding, of complement protein binding, inhibition of the shielding of the staphylococcus bacterium from recognition by a phagocytic receptor or a combination thereof.

The at least a portion of a N-terminus fibrinogen binding region may be selected from SEQ. ID NO: 3-61, preferably SEQ. ID NO: 3-30 or SEQ. ID NO: 35-61. The at least a portion of a N-terminus fibrinogen binding region may be selected from SEQ. ID NO: 3, 4, 5, 6, 7, 8, 9, 10, 11, 12, 13, 14, 15, 16, 17, 18, 19, 20, 21, 22, 23, 24, 25, 26, 27, 28, 29, 30, 35, 36, 37, 38, 39, 40, 41, 42, 43, 44, 45, 46, 47, 48, 49, 50, 51, 52, 53, 54, 55, 56, 57, 58, 59, 60, and 61. The fibrinogen binding protein may be Efb, Coa or both. The composition may further includes an antigen selected from SpA, SpA variant, Emp, EsxA, EsxB, EsaC, Eap, EsaB, Coa, vWbp, vWh, Hla, SdrC, SdrD, SdrE, IsdA, IsdB, IsdC, ClfA, ClfB, SasF Sta006, Sta011, Hla and EsxA-EsxB.

The present invention provides a pharmaceutical composition for the targeted delivery of an active agent comprising a pharmacologically effective amount of a modified extracellular fibrinogen binding protein connected to a collagen-like domain, a globular domain or both and disposed in a pharmaceutically acceptable carrier, wherein the modified extracellular fibrinogen binding protein comprises a N-terminus fibrinogen binding region that binds a fibrinogen delivering the collagen-like domain, a globular domain or both to the fibrinogen. The at least a portion of a N-terminus fibrinogen binding region may be SEQ. ID NO: 2 or SEQ. ID NO: 34. The collagen-like domain, a globular domain or both may form a hydrogel. The composition may further include an antigen selected from SpA, SpA variant, Emp, EsxA, EsxB, EsaC, Eap, EsaB, Coa, vWbp, vWh, Hla, SdrC, SdrD, SdrE, IsdA, IsdB, IsdC, ClfA, ClfB, SasF Sta006, Sta011, Hla and EsxA-EsxB.

The present invention provides a method for making a monoclonal antibody comprising the steps of: providing an effective amount of a composition comprising a modified extracellular fibrinogen binding protein having a N-terminus modified fibrinogen binding protein that does not bind fibrinogen, a C-terminus modified complement binding protein that does not bind a complement protein or both; producing an antibody pool of the modified extracellular fibrinogen binding protein, the C-terminus modified complement binding protein, or both; screening the antibody pool to detect active antibodies; wherein the active antibodies inhibit the fibrinogen binding to extracellular fibrinogen binding protein; separating the active antibodies; and adding the active antibodies to a pharmaceutically acceptable carrier.

The present invention provides a method for making a vaccine comprising the steps of: providing an effective amount of a composition comprising a modified extracellular fibrinogen binding protein having a N-terminus modified fibrinogen binding protein that does not bind fibrinogen, a C-terminus modified complement binding protein that does not bind a complement protein or both and further comprising an antigen selected from SpA, SpA variant, Emp, EsxA, EsxB, EsaC, Eap, EsaB, Coa, vWbp, vWh, Hla, SdrC, SdrD, SdrE, IsdA, IsdB, IsdC, ClfA, ClfB, SasF Sta006, Sta011, Hla and EsxA-EsxB. The N-terminus modified fibrinogen binding protein may have 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9, or 99.99% homology to SEQ ID NO: 2; SEQ ID NO: 34; or both. The at least a portion of a N-terminus fibrinogen binding region is selected from SEQ. ID NO: 3-30; from SEQ. ID NO: 35-61; or both. The at least a portion of a N-terminus modified fibrinogen binding protein is selected from SEQ. ID NO: 3, 4, 5, 6, 7, 8, 9, 10, 11, 12, 13, 14, 15, 16, 17, 18, 19, 20, 21, 22, 23, 24, 25, 26, 27, 28, 29, and 30 or from SEQ. ID NO: 35, 36, 37, 38, 39, 40, 41, 42, 43, 44, 45, 46, 47, 48, 49, 50, 51, 52, 53, 54, 55, 56, 57, 58, 59, 60, 61.

The present disclosure provides a method of vaccinating a host against staphylococcus bacterium by administering to a subject a pharmacologically effective amount of a vaccine in a pharmaceutically acceptable excipient, comprising a modified extracellular fibrinogen binding protein comprising at least a portion of a N-terminus region, at least a portion of a C-terminus region, or both, wherein the modified extracellular fibrinogen binding protein results in inhibiting the fibrinogen binding, C3 binding, or both or administering to a subject a pharmacologically effective amount of a vaccine in a pharmaceutically acceptable excipient, comprising a modified extracellular fibrinogen binding protein comprising at least a portion of a N-terminus region, at least a portion of a C-terminus region, or both, wherein the modified extracellular fibrinogen binding protein results in inhibiting the surface-bound complement protein, an antibody or both from shielding the staphylococcus bacterium from recognition by a phagocytic receptor.

The present disclosure provides a vaccine having a pharmacologically effective amount of a vaccine in a pharmaceutically acceptable excipient, comprising a modified extracellular fibrinogen binding protein comprising at least a portion of a N-terminus fibrinogen binding region, at least a portion of a C-terminus complement protein binding region, or both, wherein the modified extracellular fibrinogen binding protein results in inhibiting the fibrinogen binding, C3 binding, or both or having a pharmacologically effective amount of a vaccine in a pharmaceutically acceptable excipient, comprising a modified extracellular fibrinogen binding protein comprising at least a portion of a N-terminus fibrinogen binding region, at least a portion of a C-terminus complement protein binding region, or both, wherein the modified extracellular fibrinogen binding protein results in inhibiting the surface-bound complement protein, an antibody or both from shielding the staphylococcus bacterium from recognition by a phagocytic receptor.

The present disclosure also provides a monoclonal antibody or antigen-binding fragment thereof that can specifically bind to a portion of a extracellular fibrinogen binding protein comprising heavy and light chain variable regions that bind at least a portion of a N-terminus region of a extracellular fibrinogen binding protein that binds a fibrinogen, at least a portion of a C-terminus region of a extracellular fibrinogen binding protein that binds a complement protein, or both and results in the inhibition of the shielding of the staphylococcus bacterium from recognition by a phagocytic receptor.

One embodiment of the present disclosure provides a method for eliciting an immune response against a staphylococcus bacterium in a subject by identifying a subject having a staphylococcus bacterium; providing to the subject an effective amount of a composition comprising a modified extracellular fibrinogen binding protein (Efb) having a N-terminus binds that binds fibrinogen and a C-terminus binds a complement protein, wherein the Efb does not shield a surface-bound complement protein, an antibody or both from recognition by a phagocytic receptor.

Another embodiment of the present disclosure provides a vaccine made by combining a pharmaceutically acceptable excipient and an effective amount of a composition comprising a modified extracellular fibrinogen binding protein (Efb) having a N-terminus binds that binds fibrinogen and a C-terminus binds a complement protein, wherein the Efb does not shield a surface-bound complement protein, an antibody or both from recognition by a phagocytic receptor.

Another embodiment of the present disclosure provides a chimeric molecule of a extracellular fibrinogen binding protein (Efb) having a N-terminus binds that binds a fibrinogen; and a C-terminus that binds a complement protein, wherein the chimeric molecule can modulate complement activity, modulate antibody binding, modulate recognition by a phagocytic receptor or a combination thereof. The chimeric molecule may be capable of inhibiting or enhancing complement binding, antibody binding, recognition by a phagocytic receptor or a combination thereof.

Fibrinogen (Fg) is a plasma dimeric glycoprotein that is best known for its role in the blood coagulation cascade where thrombin proteolytically converts Fg to fibrin which then spontaneous assembles into the core of the clot. Coagulase (Coa) is a secreted staphylococcal protein and is a virulence determinant contributing to pathogenesis of staphylococcal diseases. Coa was named for its ability to support the conversion of Fg to insoluble fibrin. This activity involves Coa capturing and activating prothrombin in a non-proteolytic manner subsequently allowing the cleavage of Fg to fibrin by the activated protease. Coa also binds Fg directly independent of prothrombin. However, the molecular details underlying the Coa-Fg interaction remain elusive. The instant disclosure shows that the Fg binding activity of Coa is functionally related to that of staphylococcal Extracellular fibrinogen binding protein (Efb). In the competition ELISA assay, Coa and Efb compete with each other in binding to Fg suggesting these two staphylococcal proteins harbor similar Fg motif and are likely bind to the similar site(s) in Fg. Biochemical analyses allowed us to identify the critical residues for Fg binding in Efb and showed that the core of these residues are conserved in Fg binding motifs in Coa. This motif locates to an intrinsically disordered section of the protein and is unusually long covering 25-27 residues. Competition ELISA and isothermal titration calorimetry analyses demonstrate that Coa from Newman strain contains multiple Fg binding sites in which one locates in residues 474-505 and the others are in 5 tandem repeats which immediately follow the first binding site (residues 474-505). Binding of the Efb/Coa motif to Fg likely induces a conformational change in the plasma protein which might be the bases for the proteins ability to induce the formation of a Fg containing barrier around staphylococci that protects the bacteria from clearance by phagocytes.

### DESCRIPTION OF THE DRAWINGS

For a more complete understanding of the features and advantages of the present invention, reference is now made to the detailed description of the invention along with the accompanying figures and in which:

FIGS. 1A-1F show the full-length Efb inhibits phagocytosis of S. aureus in human plasma.

FIGS. 2A, 2B, and 2C show the simultaneous binding to Fg and C3 is essential for phagocytosis inhibition by Efb.

FIGS. 3A-3C show the purified Efb blocks phagocytosis ex vivo and in vivo.

FIGS. 4A-4D show phagocytosis inhibition by Efb is independent of complement inhibition.

FIGS. 5A-5D show that Efb attracts Fg to the bacterial surface.

FIGS. 6A-6C show that Efb prevents recognition of opsonic C3b and IgG.

FIGS. 7A-7D show endogenously produced Efb blocks phagocytosis via complex formation.

FIG. 8 shows a mechanism for phagocytosis inhibition by Efb.

FIG. 9A illustrates a schematic presentation of recombinant Coa fragments generated in this study. Coa is depicted in its secreted form Coa (27-636) lacking the signal peptide (1-26). FIG. 9B illustrates an ELISA assays of GST-tagged Coa fragments binding to immobilized Fg, Coa (Coa 27-636); Coa-N(Coa 27-310); Coa-C(Coa 311-636); Coa-R (Coa 506-636); Coa-F (Coa 311-505). FIG. 9C is a table that shows the protein concentration at which the reaction rate is half of Vmax (Km) and the goodness of fit (R²). FIG. 9D illustrates the effect of peptide Efb-O on inhibition of recombinant Coa (rCoa) binding to Fg. Increasing concentration of Efb-O were incubated with 4 nM GST-tagged Coa proteins in Fg-coated microtiter wells. Control, BSA.

FIG. 10A is a table of the Efb-O variant peptides were synthesized where each residue in the sequence is individually replaced with Ala (or Ser when the native a.a. is Ala). FIG. 10B is a plot of the Efb-O variant peptides inhibit rEfb-O (5 nM) binding to immobilized Fg in solid phase assay. Wells were coated with 0.25 μg/well Fg. Peptides (2 μM) were mixed with rEfb-O proteins (5 nM) and incubated in the Fg wells for 1 hour. FIG. 10C is a plot showing selected peptides inhibit rEfb-O binding to immobilized Fg. Increasing concentrations of Efb peptides were incubated with 5 nM rEfb-O in Fg-coated microtiter wells.

FIG. 11A is an image of a ClustalW alignment of amino acid sequence from Efb-O (Efb 68-98) and Coa from Newman strain (col-Newman). FIGS. 11B and 11C show a comparison of amino acid sequence of Efb-O with Coa 474-505 (FIG. 11B) and Coa 506-532 (FIG. 11C). FIGS. 11D and 11E show the effect of Coa peptides on inhibition of rEfb-N(Efb 30-104) (FIG. 11D) and rCoa-C (Coa 311-636) (FIG. 11E) binding to Fg by the inhibition ELISA assays.

FIG. 12A is a panel of Coa-RI variant peptides were synthesized where each residue in the sequence is individually replaced with Ala (or Ser when the native a.a. is Ala). FIG. 12B is a sCoa-RI variant peptides (50 μM) inhibit GST-tagged rCoa-C(Coa 311-636) (2 nM) binding to immobilized Fg in solid phase assay. Wells were coated with 0.25 μg/well Fg. FIG. 12C is a comparison of amino acid sequence of Efb-O with Coa-RI. FIG. 12D is a Fg-binding register of tandem repeats in Coa. Asterisks denote the residues that are important for Fg binding.

FIG. 13A is a schematic presentation of Coa peptides. FIG. 13B is a plot of the effect of Coa peptides on inhibition of rCoa-C binding to fibrinogen.

FIGS. 14A-C show a characterization of the interaction of Fg-D fragment with Coa peptides by VP-ITC.

FIG. 15 shows Coa and Efb prevent monocytic cells from adherence to fibrinogen.

FIG. 16A is a Schematic representation of DC2-Fg with fibrinogen (Fg) binding motif Efb-O.

FIG. 16B is an image of a circular dichroism (CD) spectra of DC2 and DC2-Fg. Peak at 220 nm is indicative of triple helix. FIG. 16C is plot of the integrin α1 and α2 subunit expressing C2C12 cell adhesion to DC1 (no integrin binding site), DC2 (binding site for integrins α1 and α2), DC2-Fg (DC2 with fibrinogen binding site), and collagen (multiple binding sites for integrins α1 and α2).

FIG. 16D is a graph showing fibrinogen binding to DC2, DC2-Fg, and Efb, as determined by solid phase binding assay.

### DESCRIPTION OF EMBODIMENTS

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Upon contact with human plasma, bacteria are rapidly recognized by the complement system that labels their surface for uptake and clearance by phagocytic cells. Staphylococcus aureus secretes the 16 kD Extracellular fibrinogen binding protein (Efb) that binds two different plasma proteins using separate domains: the Efb N-terminus binds to fibrinogen, while the C-terminus binds complement C3. Efb blocks phagocytosis of S. aureus by human neutrophils. In vitro, Efb blocks phagocytosis in plasma and in human whole blood. Using a mouse peritonitis model Efb effectively blocks phagocytosis in vivo, either as a purified protein or when produced endogenously by S. aureus. Mutational analysis revealed that Efb requires both its fibrinogen and complement binding residues for phagocytic escape. Using confocal and transmission electron microscopy it can be see that Efb attracts fibrinogen to the surface of complement-labeled S. aureus generating a ‘capsule’-like shield. This thick layer of fibrinogen shields both surface-bound C3b and antibodies from recognition by phagocytic receptors. This information is critical for future vaccination attempts, since opsonizing antibodies may not function in the presence of Efb. Efb from S. aureus uniquely escapes phagocytosis by forming a bridge between a complement and coagulation protein.

The present disclosure describes a novel mechanism by which S. aureus can prevent uptake by phagocytic immune cells. Specifically, the secreted S. aureus protein Extracellular fibrinogen binding protein (Efb) generates a ‘capsule’-like shield around the bacterial surface through a dual interaction with the plasma proteins complement C3b and fibrinogen. The Efb-dependent fibrinogen shield masks important opsonic molecules like C3b and antibodies from binding to phagocyte receptors. This information is critical for future vaccination attempts, since opsonizing antibodies may not function in the presence of this anti-phagocytic shield.

Phagocytosis by neutrophils is crucial to the host innate defense against invading bacteria since it leads to intracellular destruction of bacteria by production of oxygen radicals and proteolytic enzymes. Bacterial engulfment by neutrophils is strongly enhanced by the labeling or ‘opsonization’ of bacteria with plasma factors such as antibodies and complement activation products (C3b, iC3b). Complement activation takes place at the bacterial surface and is initiated by recognition molecules (Clq, Mannose Binding Lectin (MBL)) that interact with bacterial surface structures like sugars or proteins. Complement activation occurs through three different pathways (classical, lectin and alternative) that converge in the formation of C3 convertase enzymes that cleave the central complement protein C3. This cleavage step leads to massive decoration of the bacterial surface with covalently deposited C3b and iC3b molecules, which are recognized by complement receptor 1 and 3 (CR1 and CR3) on neutrophils. Complement activation proceeds by formation of C5 convertase enzymes that cleave C5 to release the potent chemoattractant C5a and C5b, which initiates formation of the membrane attack complex.

Staphylococcus aureus is an important human pathogen notorious for its ability to cause both community- and hospital-acquired diseases, ranging from mild skin infections to bacteremia, sepsis and endocarditis. Although Methicillin-resistant S. aureus (MRSA) was previously considered as an opportunistic pathogen causing hospital-acquired infections in immune-compromised patients, the emergence of the highly virulent community-associated (CA-) MRSA showed that this bacterium could also cause serious infections in otherwise healthy persons. Due to the rapid emergence of antibiotic resistance strains, alternative therapy options are now being explored. Vaccination has not been successful so far and an important reason may be the bacteria's elaborate immune evasion repertoire. Therefore, immune evasion proteins are now considered as important vaccination targets. One proposed vaccine candidate is the S. aureus Extracellular fibrinogen binding protein (Efb), a 16-kD secreted protein with a presumable role in disease pathogenesis, which is found in 85% of S. aureus strains. The secreted Efb protein consists of two functionally distinct domains: a disordered 9 kD N-terminus (Efb-N) that harbors two binding sites for fibrinogen (Fg) and a folded 7 kD C-terminus (Efb-C) that binds to the C3d domain of complement C3 (which is also present in C3b and iC3b). Although previous papers described various functions for the isolated N- and C-terminal domains of Efb, it is currently not understood why the full-length Efb protein harbors both a Fg and C3d binding site. The present disclosure shows Efb potently blocks phagocytosis of bacteria via a novel mechanism linking the complement and coagulation proteins.

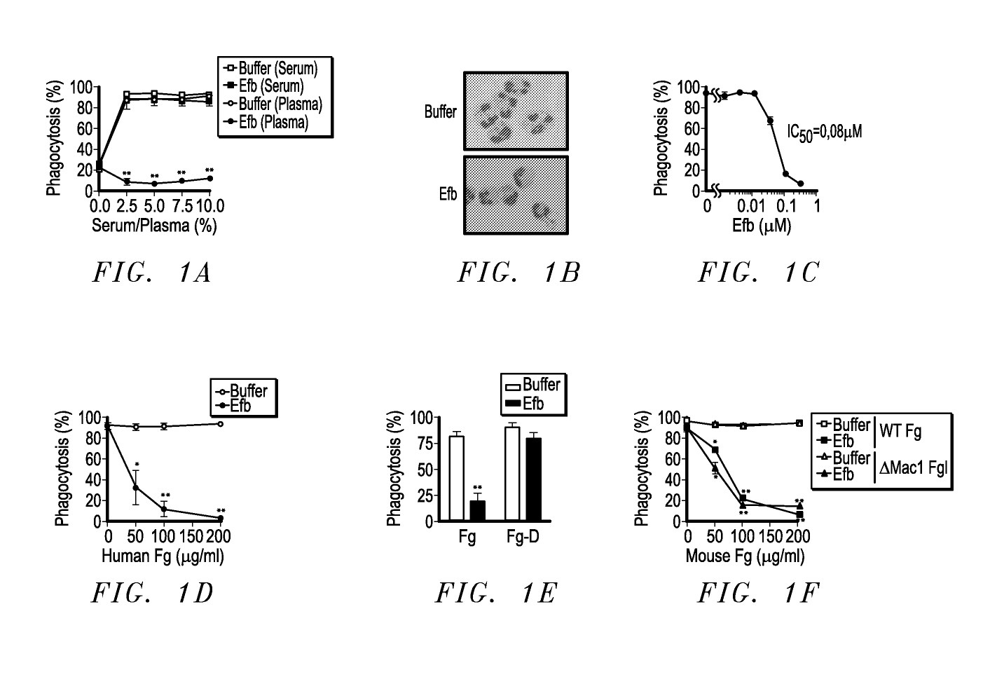

Full-length Efb inhibits phagocytosis in the presence of plasma. FIG. 1A shows phagocytosis of fluorescently labeled S. aureus by purified human neutrophils in the presence of human serum or plasma and Efb (0.5 FIG. 1B shows a histology image of human neutrophils incubated with S. aureus and 2.5% plasma in the presence or absence of Efb (0.5 Cells were stained using Diff-Quick. FIG. 1C shows dose-dependent phagocytosis inhibition by Efb in the presence of 2.5% human plasma. IC₅₀ was calculated using non-linear regression analysis, R²=0.95. FIGS. 1D-1F show phagocytosis in the presence of 5% human serum supplemented with either full-length human Fg (FIG. 1D), the D domain of human Fg (1 μM or 86 μg/ml) (FIG. 1E) or mouse Fg (WT or lacking the Mac-1 binding site) (FIG. 1F). A, C-F are mean±se of three independent experiments. B is a representative image. *P<0.05, **P<0.005 for Efb versus buffer (two-tailed Student's t-test).

The present disclosure provides potential role for full-length Efb in phagocytosis escape, fluorescently labeled S. aureus was mixed with purified human neutrophils, Efb (0.5 μM) and human serum or plasma as a source for complement and analyzed bacterial uptake by flow cytometry. In the presence of serum, Efb did not affect bacterial uptake by neutrophils (FIG. 1A). However when human plasma as a complement source was used, Efb strongly prevented phagocytosis (FIGS. 1A and 1B) and subsequent bacterial killing by neutrophils. Phagocytosis inhibition in plasma occurred in a dose-dependent fashion with a calculated IC₅₀ of 0.08 μM (FIG. 1C). Since the main difference between plasma and serum lies in the presence of coagulation proteins, it was investigated whether the observed differences in phagocytosis inhibition were caused by the fact that serum lacks Fg. The supplementation of serum with physiological concentrations of Fg led to phagocytosis inhibition by Efb (FIG. 1D). Fg is a large (340 kD) dimeric protein that comprises one central E-fragment and two lateral D-fragments. Since Efb binds to the D-fragment of Fg, it was examined if supplementing serum with Fg-D would also lead to phagocytosis inhibition by Efb. Interestingly, Efb could not block phagocytosis in the presence of Fg-D (FIG. 1E) indicating that full-length Fg is required for phagocytosis inhibition by Efb. Since Fg is a ligand for CR3 (or Mac-1) on neutrophils, it was examined whether the binding of Fg to this receptor is important for the anti-phagocytic effect of Efb. Therefore, purified Fg from wild-type mice or Fgγ^{390-396A} mice (ΔMac-1 Fg) mice that express a mutated form of Fg lacking the Mac-1 binding site but retaining clotting function. FIG. 1F shows that supplementation of human serum with both forms of mouse Fg led to inhibition by Efb, indicating that Fg binding to Mac-1 is not important for inhibition. In conclusion, Efb interferes with phagocytosis in a plasma environment and the presence of full-length Fg is required for this inhibition.

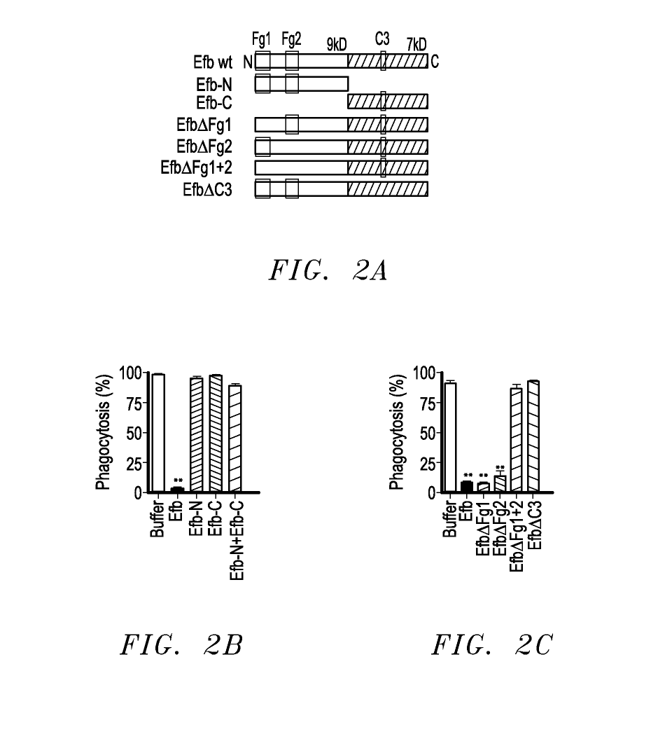

FIG. 2A shows a schematic overview of Efb mutants generated in this study. Efb is depicted in its secreted form (30-165) lacking the signal peptide (1-29). Bounding boxes indicate Fg- and C3-binding domains. The N-terminus of Efb (light grey, 9 kD) harbors two Fg binding sites named Fg1 (residues 30-67) and Fg2 (residues 68-98). The C-terminus of Efb (dark grey, 7 kD) harbors the C3 binding site (residues R131 and N138). EfbΔFg1 has deletion of residues 30-45, resulting in non-functional binding Fg1; whereas EfbΔFg2 has deletion of residues 68-76, resulting in non-functional binding Fg2. FIG. 2B shows phagocytosis of fluorescent S. aureus by human neutrophils in the presence of 5% human plasma and Efb fragments (B) or Efb mutants (C) (all at 1 μM). B&C are mean±se of three independent experiments. **P<0.005 for Efb versus buffer (two-tailed Student's t-test).

Simultaneous binding to Fg and C3 is essential for phagocytosis inhibition by Efb. To get more insight into the mechanism of inhibition, panel of Efb mutants was constructed (FIG. 2A). The individual N or C termini of Efb could not block phagocytosis in plasma (FIG. 2B). In addition, mixing the N and C terminal fragments of Efb did not markedly affect phagocytosis, indicating that full-length Efb is required. Second, mutants of full-length Efb lacking the previously characterized binding sites for Fg and C3 were generated (FIG. 2A). Three different Fg-binding mutants were created: EfbΔFg1 lacking residues 30-45, EfbΔFg2 lacking residues 68-76 and EfbΔFg1+2 lacking both these Fg binding sites. Furthermore EfbΔC3 were created in which the C3d-binding residues R131 and N138 were each replaced with a glutamic acid (E) (also known as Efb-RENE). Using ELISA's it can be seen that EfbΔFg1+2 could no longer bind Fg, while the single EfbΔFg1 and EfbΔFg2 mutants and EfbΔC3 still bound Fg. As expected, all mutants except EfbΔC3 bound to C3b. Next, these mutants in the neutrophil phagocytosis assay were compared in the presence of human plasma. EfbΔFg1+2 and EfbΔC3 could no longer block phagocytosis (FIG. 2C), indicating that a simultaneous interaction with both Fg and complement C3 (products) is essential for the anti-phagocytic action of Efb. The finding that EfbΔFg1 and EfbΔFg2 were still active indicates that Efb requires only one of its two Fg binding sites to block phagocytosis.

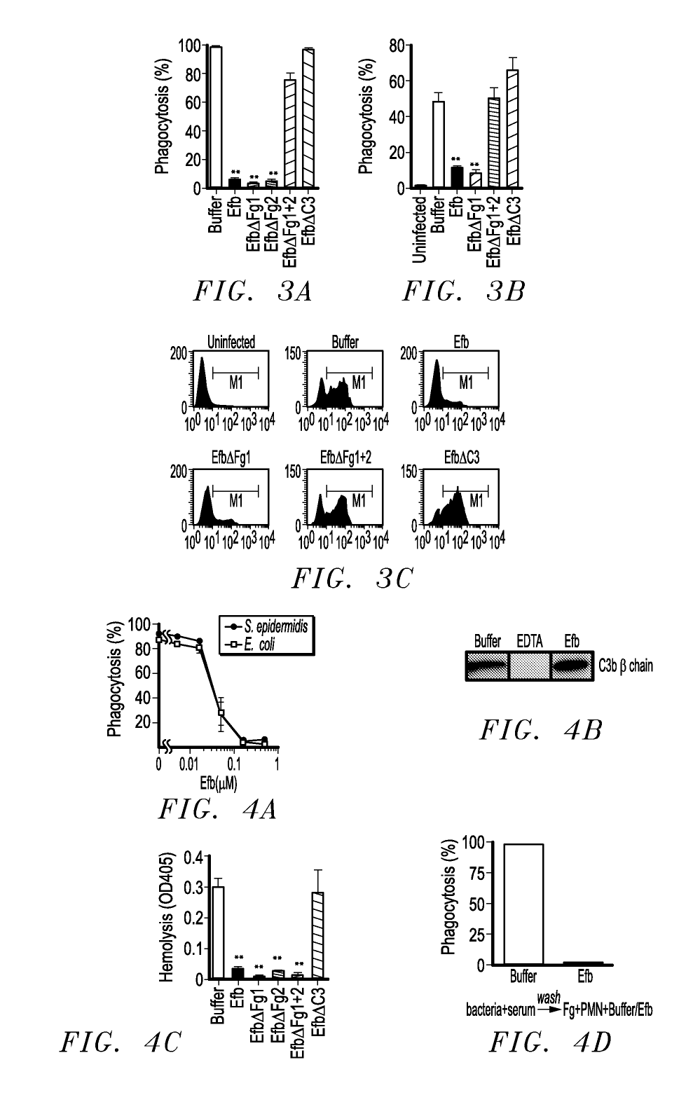

FIG. 3A shows Ex vivo phagocytosis of fluorescent S. aureus incubated with 50% human whole blood and Efb (1 μM). Neutrophils were gated based on forward and side scatter properties. FIG. 3B shows In vivo phagocytosis of fluorescent S. aureus by human neutrophils in the mouse peritoneum. Neutrophils were attracted to the peritoneal cavity using carrageenan (i.p.) and subsequently challenged with 10⁸ heat-inactivated fluorescent S. aureus and Efb (1 μM) for 1 hour. The peritoneal lavage was collected and neutrophil phagocytosis was analyzed by flow cytometry. Neutrophils were gated based on Gr-1 expression. The mouse studies were carried out three times. 3 mice per group were used and the cells of these 3 mice were pooled for phagocytosis analysis. FIG. 3C shows a representative histograms of FIG. 3B. A, B are mean±se of three independent experiments. *P<0.05, **P<0.005 for Efb versus buffer (two-tailed Student's t-test).

Efb blocks phagocytosis ex vivo and in vivo. To study whether Efb can also block phagocytosis in a natural environment, its activity in ex vivo and in vivo was examined using phagocytosis models. In an ex vivo human whole blood model, fluorescent S. aureus was incubated with 50% human whole blood and Efb. After 25 minutes, neutrophil phagocytosis was analyzed by flow cytometry. Full-length Efb potently blocked phagocytosis by human neutrophils in whole blood (FIG. 3A) and that this inhibition depends on the interaction of Efb with both Fg and C3. Phagocytosis of S. aureus in an in vivo mouse peritonitis model was examined. To this end, mice were treated with carrageenan (i.p.) to induce neutrophil infiltration into the peritoneal cavity and subsequently challenged with 10⁸ heat-inactivated fluorescent S. aureus in the presence or absence of Efb (1 μM). One hour later, mice were sacrificed and the peritoneum was lavaged with sterile PBS. Neutrophils were stained and phagocytosis of fluorescent bacteria was analyzed by flow cytometry. It can be seen that Efb blocked phagocytosis in the peritoneum (FIGS. 3B and 3C). Efb mutants showed that inhibition of phagocytosis in vivo also depends on the Fg and C3 binding domains of Efb.

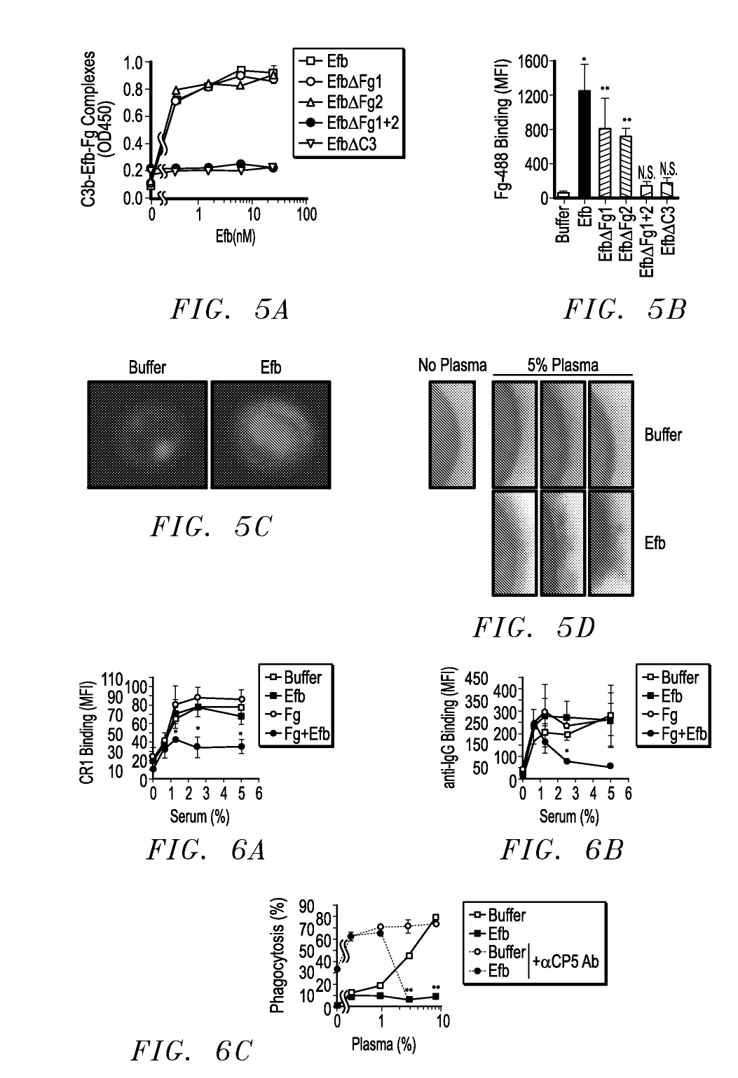

FIG. 4A shows phagocytosis of fluorescently labeled S. epidermidis and E. coli by purified human neutrophils in the presence of human plasma (5%) and Efb. FIGURE shows 4B immunoblot detecting surface-bound C3b after incubation of S. aureus with 5% human plasma in the presence of 5 mM EDTA or 0.5 μM Efb. Blot is a representative of 3 independent experiments. FIG. 4C shows alternative pathway hemolysis of rabbit erythrocytes in 5% human plasma and Efb (mutants) (1 μM). Bars are the mean±se of three independent experiments. **P<0.005 for Efb versus buffer (two-tailed Student's t-test). FIG. 4D shows phagocytosis with a washing step. Fluorescent S. aureus was first incubated with 5% serum to deposit complement. Bacteria were washed and subsequently mixed with neutrophils and Fg in the presence or absence of Efb (0.5 μM).

Phagocytosis inhibition by Efb is independent of complement inhibition. Studies shown above indicate that Efb requires an interaction with both complement and Fg to block phagocytosis. To study whether Efb also interacts with S. aureus specifically, it was analyzed whether purified Efb can block phagocytosis of other bacteria as well. Fluorescent S. epidermidis or E. coli were mixed with human plasma and phagocytosis by neutrophils was evaluated. Efb potently inhibits the uptake of these bacteria as well, indicating that Efb can block phagocytosis independently of S. aureus (FIG. 4A). The C-terminal domain of Efb is a complement inhibitor that inactivates C5 convertases to prevent cleavage of C5. Efb-C did not affect C3b labeling of bacteria in conditions where all complement pathways are active. However, since the effects of Efb on complement were performed with serum instead of plasma, it was examined whether full-length Efb might affect C3b labeling of bacteria in a plasma environment. S. aureus was incubated with human plasma and Efb and quantified surface-bound C3b using immunoblotting. As a control, EDTA was added to prevent activation of all complement routes (which are calcium and magnesium dependent). Lower amounts of C3b was not found on the bacterial surface in the presence of Efb compared to buffer (FIG. 4B), indicating that Efb does not interfere with C3b labeling in plasma. Subsequently, the inhibition of C5 convertases by Efb (mutants) in plasma using an alternative pathway hemolytic assay was examined. Rabbit erythrocytes were incubated with human plasma and C5 cleavage was measured by means of C5b-9 dependent lysis of erythrocytes.

In conjunction with previous results in serum, it can be see that all Efb mutants except for EfbΔC3 inhibited C5 cleavage in plasma (FIG. 4C). Since this inhibition exclusively depends on the C-terminal domain (all Fg binding mutants of Efb could still block C5 cleavage), this proves that interference with C5 cleavage is at least not sufficient for phagocytosis inhibition by Efb. To further show that the effects of Efb on complement activation are dispensable for phagocytosis inhibition a washing step was added to the phagocytosis assay. Bacteria were first incubated with serum (in the absence of Efb) to deposit C3b. After washing away unbound serum proteins (including C5a), these pre-opsonized bacteria were incubated with Fg and neutrophils. In this assay, Efb could potently block phagocytosis (FIG. 4D). In conclusion, these results indicate that the anti-phagocytic activity of Efb is not related to its complement-inhibitory effect.

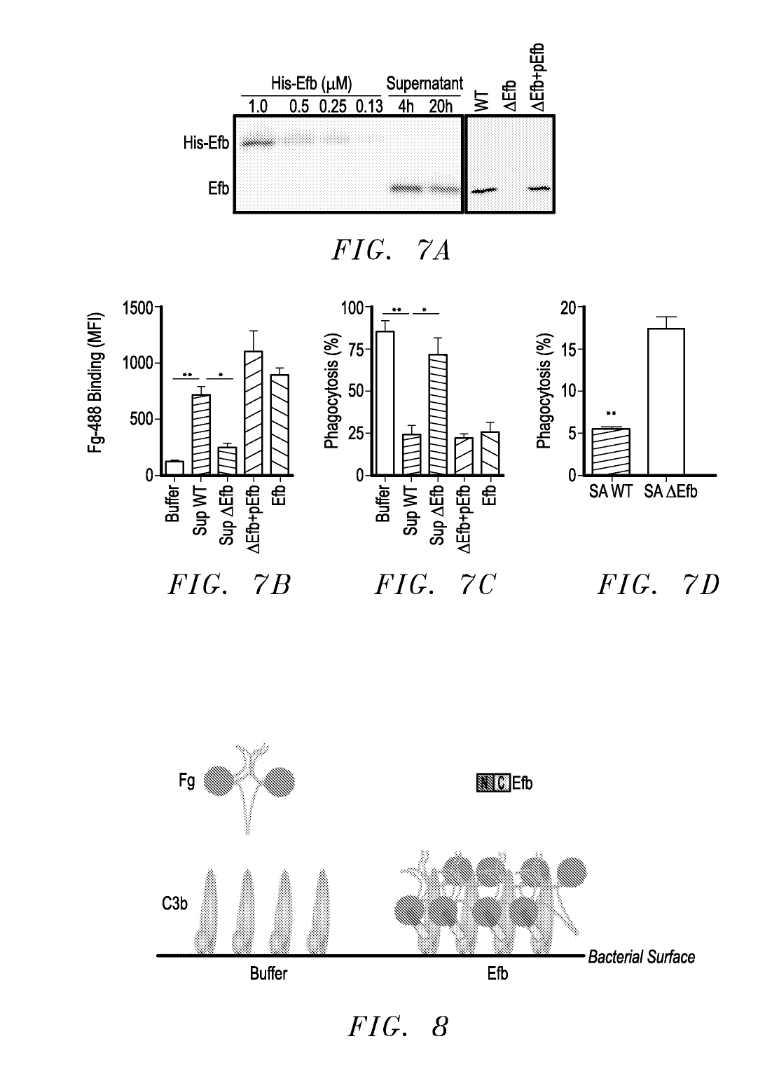

FIG. 5 shows an ELISA showing that Efb can bind Fg and C3b at the same time. C3b-coated microtiter wells were incubated with Efb (mutants) and, after washing, incubated with 50 nM Fg that was detected with a peroxidase-conjugated anti-Fg antibody (Abcam). Graph is a representative of two independent studies performed in duplicate. FIG. 5B shows binding of Alexa488-labeled Fg (60 μg/ml) to serum-opsonized S. aureus in the presence of Efb (mutants) (0.5 μM) Graph represents mean±se of three independent experiments. *P<0.05, **P<0.005 for Efb versus buffer (two-tailed Student's t-test). N.S. is not significant. FIG. 5C shows confocal analysis of samples generated in B (representative images). FIG. 5D shows TEM pictures of S. aureus incubated with 5% human plasma in the absence or presence of Efb (0.5 μM).

Efb covers S. aureus with a shield of Fg. To determine whether Efb might bind to C3b-labeled bacteria and then attract Fg to the surface, full-length Efb binding to Fg and C3b at the same time. C3b-coated microtiter plates were incubated with Efb and, after a washing step, treated with Fg. FIG. 5A shows that Efb is able to form a complex with C3b and Fg. Also, the EfbΔFg1 and EfbΔFg2 mutants could still form Fg-C3b complexes. In contrast, complex formation was not detected for the mutants that lack either both Fg (EfbΔFg1+2) or the C3 binding domains (EfbΔC3) (FIG. 5A). Then Efb binding and attracting Fg to pre-opsonized bacteria was examined. Therefore, S. aureus was pre-opsonized with human serum to deposit complement and subsequently incubated with Efb. After washing, bacteria were incubated with Alexa-488 conjugated Fg. Using both flow cytometry and confocal microscopy it can be seen that that Efb mediates Fg binding to pre-opsonized bacteria (FIGS. 5B, 5C). Consistent with the ELISA data for complex formation, no Fg binding was detected in the presence of EfbΔFg1+2 or EfbΔC3. Confocal analyses indicated that Efb covers the complete bacterial surface with Fg (FIG. 5C). Using Transmission Electron Microscopy this Fg layer created by Efb and be seen in more detail. After incubation of S. aureus with plasma and Efb, a diffuse outer layer formed around the bacteria (FIG. 5D). Altogether these studies show that Efb binds to C3b on the bacterial surface and subsequently attracts Fg forming a shield around the bacterial surface.

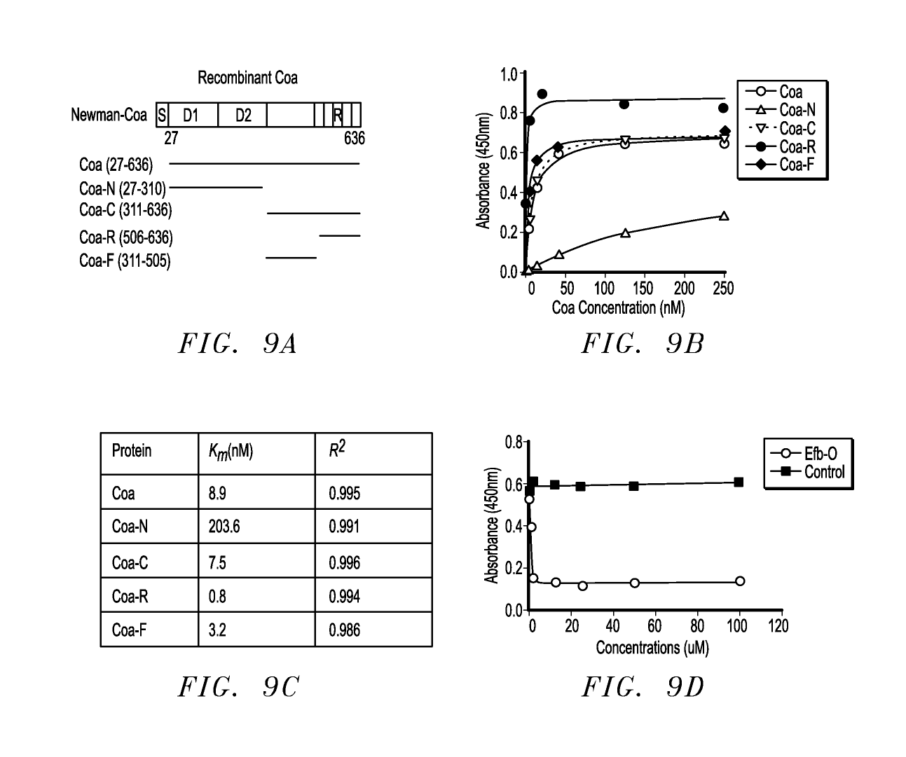

Flow cytometry assay detecting binding of soluble CR1 (FIG. 6A) or anti-IgG antibody (FIG. 6B) to pre-opsonized S. aureus in the presence of buffer, Efb (0.5 μM) and/or Fg (200 μg/ml).

FIG. 6C shows Efb inhibits phagocytosis of encapsulated S. aureus by human neutrophils. FITC-labeled S. aureus strain Reynolds (high capsule CP5 expressing strain) was incubated with human plasma and/or Efb (0.5 μM) in the presence (dotted line) or absence (solid line) of polyclonal rabbit anti-CP5 antibody. All figures represent the mean±se of three separate experiments. *P<0.05, **P<0.005 for Efb+Fg versus buffer (A,B) or Efb versus buffer (for dotted lines) (two-tailed Student's t-test).

Efb blocks recognition of C3b and IgG on the surface. Since Efb covers bacteria with a shield of Fg, which would frustrate the binding of phagocytic receptors to their ligands on the bacterial surface using flow cytometry, it was first analyzed whether C3b-labeled bacteria were still recognized by CR1. Pre-opsonized S. aureus was incubated with soluble CR1 in the presence of Fg and Efb. Clearly, binding of CR1 to pre-opsonized bacteria was blocked by the presence of both Fg and Efb (FIG. 6A). Addition of Fg or Efb alone did not affect CR1 binding. Next, it was investigated whether the Fg shield specifically blocks C3b-CR1 interactions or whether it also disturbs the binding of neutrophil Fc receptors to opsonic antibodies. To analyze this, it was determined whether the Fc part of bacterium-bound IgG could still be recognized by specific antibodies and found that incubation of pre-opsonized bacteria with Efb and Fg disturbs recognition of the antibody Fc domain on the surface (FIG. 6B), suggesting that Fc receptors can no longer recognize their target. This information is crucial for future vaccine development since opsonic antibodies against S. aureus may not function when Efb hides these antibodies underneath an Fg shield. To further prove that Efb functionally blocks opsonization, phagocytosis of an encapsulated S. aureus strain in the presence or absence of anti-capsular antibodies was analyzed. The encapsulated S. aureus strain Reynolds was grown for 24 hours in Columbia agar supplemented with 2% NaCl (for optimal capsule expression) and subsequently labeled with FITC. Capsule expression after FITC-labeling was confirmed using specific antibodies. In low plasma concentrations (0-1%), it was observed that anti-capsular antibodies caused a 6-fold increase in phagocytic uptake of encapsulated S. aureus (FIG. 6C). At these plasma concentrations, Efb could not block phagocytosis. However at higher plasma concentrations (3% and more), Efb potently impeded phagocytosis in the presence of anti-capsule antibody (FIG. 6C). These data support our idea that the Fg shield created by Efb prevents recognition of important opsonins like C3b and IgG, also in the context of a capsule-expressing strain that is targeted by specific antibodies.

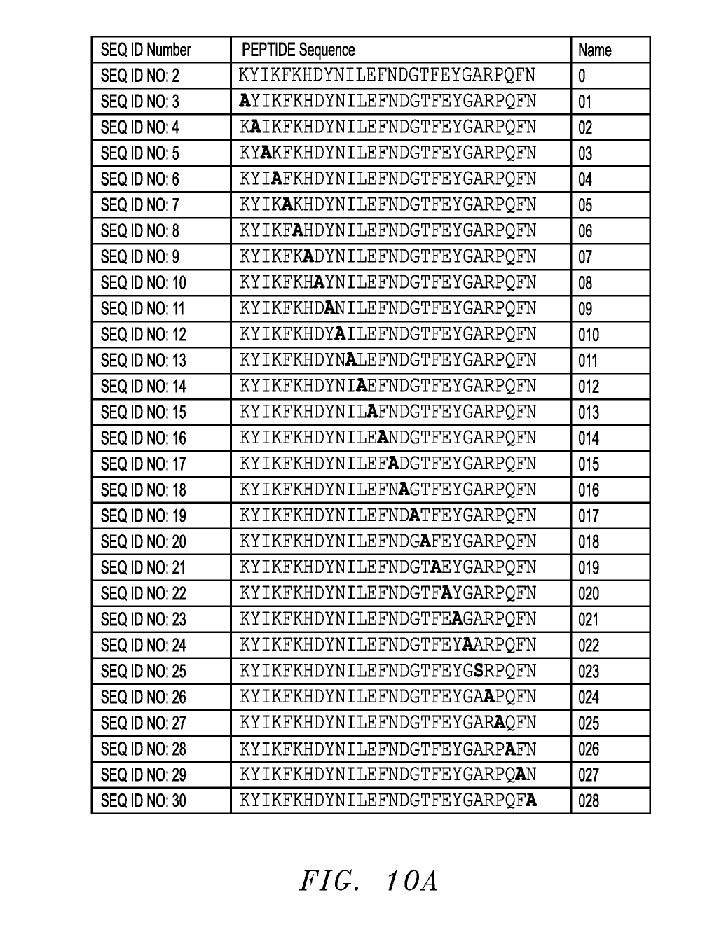

FIG. 7A left shows immunoblot detecting Efb in 4 h and 20 h culture supernatants of S. aureus Newman; fixed concentrations of His-tagged Efb were loaded as controls. FIG. 7A right shows immunoblot of 4 h culture supernatants of S. aureus Newman (WT), an isogenic Efb deletion mutant (ΔEfb) and its complemented strain (ΔEfb+pEfb). Blots were developed using polyclonal sheep anti-Efb and Peroxidase-labeled donkey anti-sheep antibodies. Blot is a representative of two independent experiments. FIG. 7B shows flow cytometry analysis of the binding of Alexa488-labeled Fg to pre-opsonized S. aureus in the presence of 4 h culture supernatants (2-fold diluted) or purified Efb (250 nM). FIG. 7C shows In vitro phagocytosis of fluorescently labeled S. aureus by purified human neutrophils. Pre-opsonized S. aureus was first incubated with 4 h culture supernatants (2-fold diluted) or purified Efb (250 nM) and subsequently mixed with Fg and neutrophils. FIG. 7D shows In vivo phagocytosis of GFP-expressing wild-type or Efb-deficient S. aureus strains by neutrophils in the mouse peritoneal cavity. Neutrophils were attracted to the peritoneal cavity using carrageenan (i.p.) and subsequently injected with 300 μl of GFP-expressing wild-type (SA WT) or Efb-deficient (SAΔEfb) S. aureus strains during the exponential phase of growth. The peritoneal lavage was collected 1 h thereafter and neutrophil phagocytosis was analyzed by flow cytometry. Neutrophils were gated based on Gr-1 expression. Graphs in B-D represent mean±se of three independent experiments. *P<0.05, **P<0.005 for Buffer versus WT Sup or WT (Sup) versus ΔEfb (Sup) (two-tailed Student's t-test).

Endogenous Efb blocks phagocytosis in vitro and in vivo. To study whether endogenous expression of Efb leads to impaired phagocytosis of S. aureus via complex formation, the analyses was extended with (supernatants of) an isogenic Efb-deletion mutant in S. aureus Newman. First immunoblotting was performed to semi-quantify the production levels of Efb in liquid bacterial culture supernatants. Supernatants of wild-type (WT) S. aureus Newman were subjected to Immunoblotting and developed using polyclonal anti-Efb antibodies (FIG. 7A). Efb expression in the supernatant was quantified using ImageJ software and compared with fixed concentrations of purified (His-tagged) Efb using linear regression analysis (R²=0.986). Efb levels in 4 hours and 20 hours supernatants contained 1.1 μM and 0.9 μM Efb respectively. Although the Efb levels in strain Newman are suspected to be higher than in other S. aureus strains (up to 10-fold, due to a point mutation in the SaeR/S regulatory system that drives expression of immune evasion genes), the fact that these levels are >10 times higher than the calculated IC₅₀ needed for phagocytosis inhibition (0.08 μM, FIG. 1C), suggests that Efb concentrations required for phagocytosis inhibition can be reached in vivo. In a separate Immunoblot, the presence of Efb was checked in 4 hours supernatants of the WT, Efb-deficient (ΔEfb) and the complemented strain (ΔEfb+pEfb) confirming the lack of Efb expression in the mutant (FIG. 7A). Next these supernatants was used to study whether endogenous Efb can mediate C3b-Fg complex formation on the bacterial surface. S. aureus was first incubated with serum to deposit C3b, then mixed with bacterial supernatants and subsequently incubated with fluorescently labeled Fg. Whereas WT supernatants attracted Fg to the surface of pre-opsonized bacteria, Efb-deficient supernatants did not mediate complex formation (FIG. 7B). This phenotype was restored in the complemented strain. Then it was studied whether endogenous Efb could inhibit phagocytosis by neutrophils in vitro. Therefore it was repeated the latter study (but using fluorescent bacteria and unlabeled Fg) and subsequently mixed the bacteria with human neutrophils. That supernatants of WT and complemented strains were found to inhibit phagocytosis, while Efb-deficient supernatants did not influence this process (FIG. 7B). To mimic bacterial phagocytosis during a natural infection, carrageenan-treated mice were injected i.p. with GFP-expressing WT S. aureus or the Efb-deficient mutant in their original broth culture and sacrificed 1 h thereafter. Mice were subjected to peritoneal lavage and the percentage of neutrophils with internalized staphylococci was determined by flow cytometry. As depicted in FIG. 7D, the Efb-deficient S. aureus strain was phagocytosed by neutrophils to a significantly higher extent than the WT strain despite of the fact that the amount of inoculated bacteria was comparable in both groups (app. 2×10⁷). These observations demonstrate that the levels of Efb produced by S. aureus are sufficient for preventing phagocytosis in vivo.

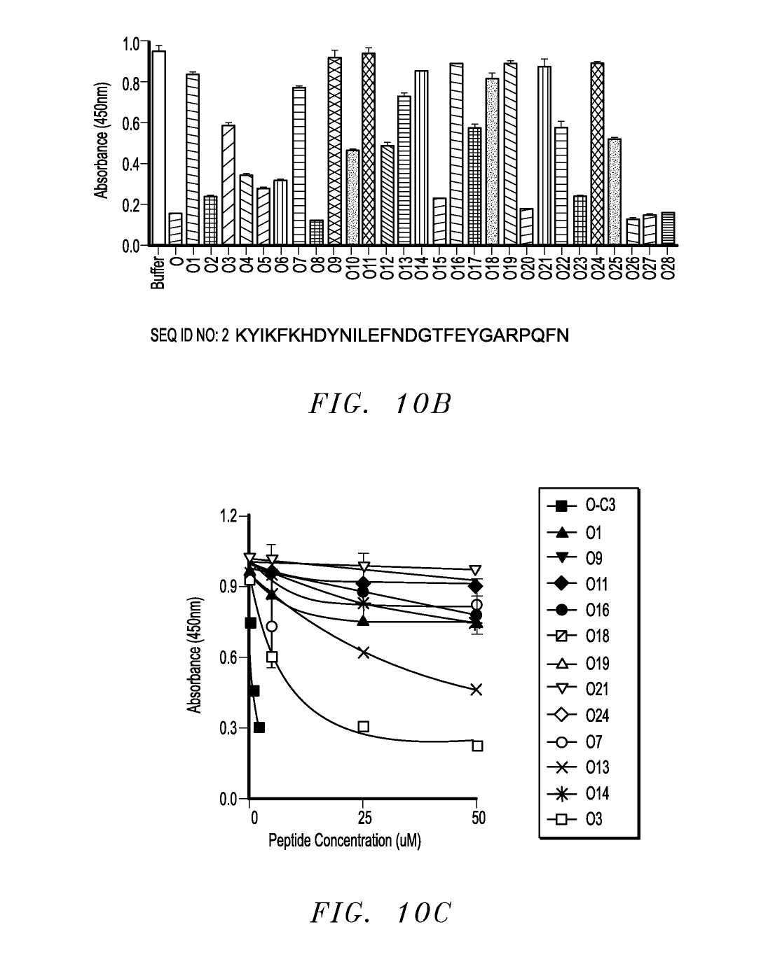

FIG. 8 shows a schematic picture of the phagocytosis escape mechanism by Efb. Left, Complement activation on the bacterial surface results in massive labeling of S. aureus with C3b molecules, while Fg stays in solution. Right, S. aureus secretes Efb, which binds to surface-bound C3b via its C-terminal domain (colored yellow). Using its N-terminus (green), Efb attracts Fg to the bacterial surface. This way, S. aureus is covered with a shield of Fg that prevents binding of phagocytic receptors to important opsonins like C3b and IgG.

The coagulation system has a dual role in the host defense against bacterial infections. On one hand, coagulation supports innate defenses by entrapment and killing of invading bacteria inside clots or via the formation of small antibacterial and pro-inflammatory peptides. On the other hand, bacterial pathogens can utilize coagulation proteins to protect themselves from immune defenses. It was found that S. aureus effectively protects itself from immune recognition by secreting Efb that specifically attracts Fg from the solution to the bacterial surface creating a capsule-like shield (FIG. 8). To accomplish this, Efb forms a multi-molecular complex of soluble Fg and surface-bound C3b. The fact that the levels of C3b at the bacterial surface are high and that Fg is an abundant plasma protein (1.5-4.0 g/L) makes this a very efficient anti-phagocytic mechanism. The Fg shield created by Efb effectively protects S. aureus from recognition by phagocyte receptors. The attracted Fg was found not only to block the binding of C3b to its receptor, but also hides the important opsonin IgG underneath the Fg shield. This information is critical for vaccine development against S. aureus. Generation of protective ‘opsonizing’ antibodies recognizing S. aureus surface structures was considered to be an important goal of vaccination. However, these antibodies will not function if they are protected underneath a layer of Fg. Including Efb in future vaccines might be beneficial as it could prevent formation of this anti-phagocytic shield and enhance the function of opsonizing antibodies. The fact that Efb is conserved among S. aureus strains may make it a suitable vaccine candidate.

Next to Efb, S. aureus secretes two other proteins that specifically interact with the coagulation system: the S. aureus ‘coagulases’ named Coagulase and Von Willebrand factor binding protein are secreted proteins that activate prothrombin in a nonproteolytic manner and subsequently convert Fg into fibrin. Thereby, coagulases embed bacteria within a network of fibrin, protecting them from immune recognition and facilitate formation of S. aureus abscesses and persistence in host tissues. Coagulase and Efb are expressed at the same time during infection since they are both regulated by the SaeRS regulator for secreted (immune evasion) proteins. Efb is highly important for proper functioning of Coagulase since Efb can attract Fg to the bacterial surface. This way, Efb may aid Coagulase-dependent fibrin formation to occur close to the bacterial surface instead of in solution. Nevertheless our studies also indicate that Efb can block phagocytosis in the absence of prothrombin and Coagulase. However, in a more complex environment the anti-phagocytic mechanisms of Efb and S. aureus Coagulase might work synergistically. Furthermore, it seems tempting to speculate that the ability of Efb to attract Fg to the bacterial surface is also beneficial in other infection processes like adhesion. Since, Fg is an important constituent of the extracellular matrix (ECM), Efb might also facilitate binding of C3b-opsonized bacteria to the ECM. In fact, Efb was previously classified as an adhesion molecule belonging to the group of SERAMs (secreted expanded repertoire adhesive molecules). However, as a secreted protein, Efb cannot facilitate bacterial adhesion if it solely binds to Fg in the ECM without interacting with the bacterial surface. Binding to C3b-labeled bacteria via the Efb C-terminus might therefore be crucial for effective bacterial adhesion to Fg.

The pathogenic potential of S. aureus is a result of its versatile interactions with multiple host factors, evidenced by the fact that it can survive at multiple sites of the body causing a wide range of infections. At most body sites, S. aureus has to deal with cellular and humoral components of the immune system. However, increasing evidence now suggests that S. aureus protects itself from immune defense by forming abscess communities surrounded by capsule-like structures that prevent neutrophil invasion. Our study implicates that Efb might be crucial in the formation of these capsules. Furthermore, our whole blood assays shows that Efb may also play an important role in S. aureus survival in the blood allowing it to spread to other sites of the body. Previous studies using animal models have highlighted the critical role of Efb in S. aureus pathogenesis. For instance, Efb delays wound healing in a rat wound infection model and is important for S. aureus pneumonia and abscess formation in kidneys. Our in vivo studies corroborate the in vitro findings and suggest that complex formation can occur under physiological conditions in vivo, however, the available mouse models do not closely mimic this process during clinical infections in humans. Efb is produced in later stages of bacterial growth, thus the bacteria need time to produce Efb before they come into contact with neutrophils. Since neutrophils need to be recruited from the blood to the site of the infection, there normally is time for Efb production and complex formation, especially in the human host where an infection starts with a low number of bacteria. In contrast, in available mouse models the timing is much different as a high inoculum (up to 10⁸ bacteria) is required to establish an infection and these high numbers of bacteria trigger a strong inflammatory response resulting in that the bacteria are already phagocytized before Efb is produced. For this reason, the bacteria was mixed with their supernatants to ensure the presence of endogenous Efb during the course of the studies and chosen a model in which neutrophils are already attracted to the infection site to focus on the anti-phagocytic activity of the molecule. Future studies are needed to design and execute appropriate animal studies that overcome the limitations of current models and better reflect the clinical situation. The present disclosure provides that full-length Efb can inhibit phagocytosis in a unique way through its dual interaction with complement and Fg. Our studies indicate that Efb is a highly effective immune escape molecule that blocks phagocytosis of S. aureus in vivo.

Fg is a major plasma dimeric glycoprotein composed of three polypeptides, Aα, Bβ, and γ. Fg is best known for its role in the later stages in the blood coagulation cascade where thrombin proteolyticly converts Fg to fibrin which then spontaneous assemble into the ultrastructural core of the clot. However, Fg is also a critical participant in a number of different physiological processes such as thrombosis, wound healing, and angiogenesis and in innate immune defense against pathogens. A role for Fg in inflammation is evident from analysis of Fg knockout mice, which exhibit a delayed inflammatory response as well as defects in wound healing. Furthermore the fibrinopeptides, generated by thrombin cleavage of Fg, are potent chemoattractants, which can act as modulators in inflammatory reactions. A genetically engineered mouse expressing a mutant form of Fg that is not recognized by the leukocyte integrin α_{M}β₂ has profound impediment in clearing S. aureus following intraperitoneal inoculation. This study highlights the importance of Fg interactions with the lekocyte integrin α_{M}β₂/Mac-1/complement receptor 3 in the clearance of staphylococci. Fg also interacts with the complement system and modulates complement dependent clearance of bacteria.

Recent studies of some of the secreted Fg binding S. aureus VFs point to yet another mechanism of Fg dependent inhibition of bacterial clearance. In a mouse model of S. aureus abscess formation, Fg accumulates and is co-localized with coagulase (coa) and von Willebrand factor binding protein (vWbp) within the staphylococcal abscess lesions. The profound amount of Fg in the periphery of the abscess forms a capsule-like structure that borders the uninfected tissue and prevents phagocytes from accessing and clearing bacteria in the center of the abscess. Furthermore, it was recently reported that Efb can assemble a Fg protective shield around the bacteria that results in impaired clearance of the organism. Efb is a 16-kD secreted protein found in 85% of S. aureus strains. The secreted Efb protein consists of two functionally distinct domains: a disordered N-terminus that harbors two related binding sites for Fg and a folded C-terminus that binds to the C3d domain of complement C3. To assemble a Fg shield Efb has to bind to C3b deposited on the surface of the bacteria via its C-terminal domain whereas the N-terminal Efb section recruits Fg.

Coagulase (Coa) is an “old” S. aureus hall mark protein best known for its ability to induce blood/plasma coagulation which allows the classification of the staphylococcal genus into coagulase positive and negative species. More recent studies have shown that Coa is a critical virulence factor in some staphylococcal diseases. Coa dependent blood coagulations is initiated by the staphylococcal protein activating the zymogen prothrombin by insertion of the Ile¹-Val² N-terminus of Coa into the Ile¹⁶ pocket of prothrombin, inducing a conformational change and a functional active site in the serine protease. This activation process does not involve proteolytic cleavage of prothrombin which is required in physiological blood coagulation. The Coa/prothrombin complex then recognizes Fg as a specific substrate and converts it into fibrin. The crystal structure of Coa/prothrombin complex reveals that the exosite 1 of α-thrombin, the Fg recognition site, is blocked by D2 domain of Coa. This information raises questions concerning the nature of Fg recognition and subsequent cleavage by the complex. Coa can interact with Fg directly without the aid of prothrombin and this interaction site(s) was tentatively located to the C-terminus of Coa. The C-terminal region of Coa is comprised of tandem repeats of a 27-residue sequence that is relatively conserved among strains but the numbers of repeats varies from 5 to 8 in different strains. The Fg-binding activity of Coa was characterized and show that Coa contains multiple copies of a Fg binding motif that is structurally and functionally related to the Fg binding motifs in Efb. The interaction of this common motif with Fg is analyzed in some detail.

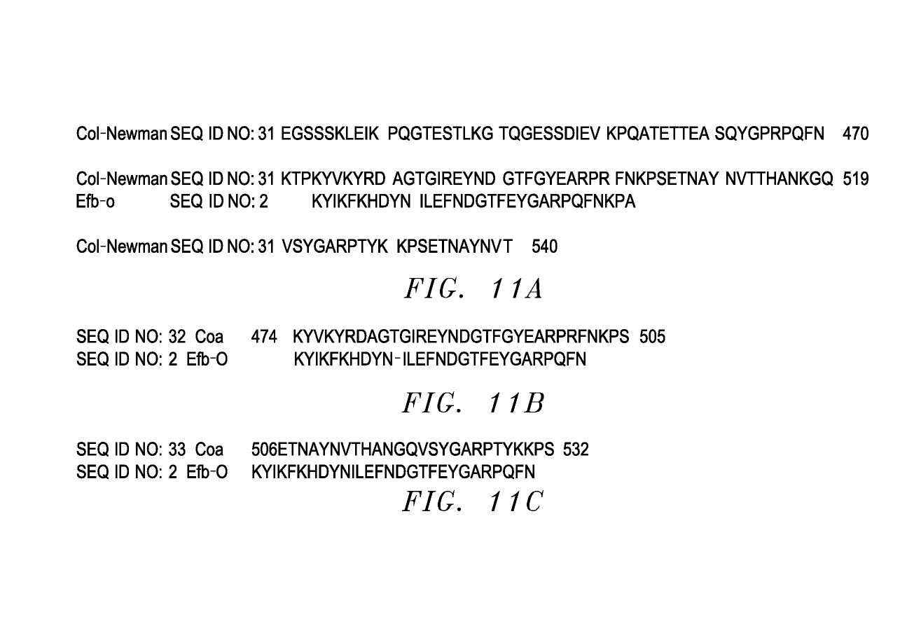

FIG. 9A illustrates a schematic presentation of recombinant Coa fragments generated in this study. Coa is depicted in its secreted form Coa (27-636) lacking the signal peptide (1-26). The N-terminus of Coa (Coa-N; Coa 27-310) constitutes D1D2 prothrombin binding domain. The C-terminus of Coa (Coa-C; Coa 311-636) includes the central region and the tandem-repeat region. The Coa-C further divides into two parts, the Coa-R is corresponding to the tandem-repeat region covering residue 506-636, and the Coa-F fragment covering residues 311-505. S, signal peptide. FIG. 9B illustrates an ELISA assays of GST-tagged Coa fragments binding to immobilized Fg. Orange, Coa (Coa 27-636); purple, Coa-N (Coa 27-310); blue, Coa-C(Coa 311-636); red, Coa-R (Coa 506-636); green, Coa-F (Coa 311-505). FIG. 9C is a table that shows the protein concentration at which the reaction rate is half of Vmax (Km) and the goodness of fit (R²). FIG. 9D illustrates the effect of peptide Efb-O on inhibition of rCoa binding to Fg. Increasing concentration of Efb-O were incubated with 4 nM GST-tagged Coa proteins in Fg-coated microtiter wells. Control, BSA.

Staphylococcal Coagulase contains multiple Fibrinogen binding sites. With the goal to identify the Fg-binding motifs in Coa we first sought to locate the Fg-binding site(s) in the protein. To this end, a panel of recombinant proteins covering different segments of Coa (FIG. 9A) was constructed and examined their Fg-binding activities in an ELISA-type binding assay. Earlier observations that Coa interacts with Fg primarily through the disordered C-terminal part of the protein (Coa-C, corresponding to residues Coa 27-636) were confirmed. Fg-binding to recombinant Coa-C is a concentration dependent process that exhibits saturation kinetics and shows half maximum binding at 7.5 nM (FIG. 9B). The tandem repeat region of Coa (fragment Coa-R, corresponding to residues Coa 506-636) binds to Fg in a similar way but with a higher apparent affinity (0.8 nM) compared to that of the whole C terminus (Coa-C). A recombinant protein containing the segment between the D1D2 domain and Coa-R was therefore constructed (fragment Coa-F, corresponding to residues Coa 311-505) and that recombinant Coa-F also binds Fg (FIG. 9B). The N-terminal D1D2 domain of Coa (Coa-N) that contains the prothrombin binding activity also interacts with Fg. However, the apparent affinity observed for Coa-N binding to Fg was much lower than that exhibited by Coa-C and the Fg-binding activity of the Coa-N was therefore not further examined in this study.

The fibrinogen binding activities in Coagulase and Efb are functionally related. Efb is another secreted Fg-binding small protein produced by S. aureus where the Fg-binding activity has been located to a disordered region in the N-terminal part of the protein. Two related Fg-binding segments in Efb named Efb-O (corresponding to Efb 68-98) and Efb-A (corresponding to Efb 30-67) were identified. The Efb-O segment was determined to have a higher affinity for Fg compared to Efb-A but that the two motifs likely bound to the same region in Fg since recombinant Efb-O (rEfb-O) effectively inhibited rEfb-A binding to the host protein. Because the Fg-binding activities in Efb and Coa are both located to disordered regions and both proteins can induce a protective Fg containing barrier we explored the possibility that the Fg-binding motifs in the two proteins are functionally related. To this end it was used a competition ELISA where the binding of recombinant Coa to Fg coated wells was quantitated in the presence of increasing concentrations of the synthetic peptide Efb-O (sEfb-O) that mimics the high affinity Fg-binding motif in Efb. Peptide sEfb-O effectively inhibited recombinant Coa binding to Fg (FIG. 9D), suggesting that Coa and Efb are functionally related and that the dominant Fg-binding motifs found in the two proteins likely bind to the same or overlapping sites in Fg.

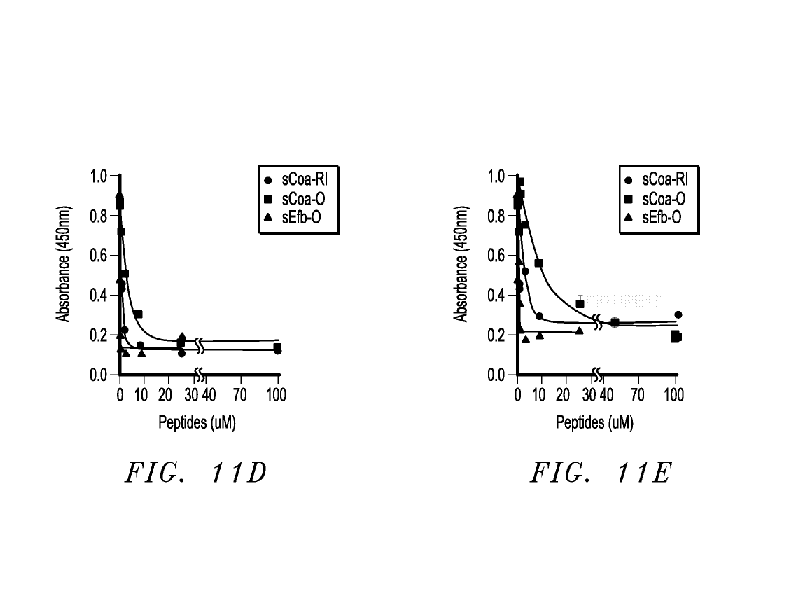

FIG. 10A is a table of the Efb-O variant peptides were synthesized where each residue in the sequence is individually replaced with Ala (or Ser when the native a.a. is Ala). FIG. 10B is a plot of the Efb-O variant peptides inhibit rEfb-O (5 nM) binding to immobilized Fg in solid phase assay. Wells were coated with 0.25 μg/well Fg. Peptides (2 μM) were mixed with rEfb-O proteins (5 nM) and incubated in the Fg wells for 1 hour. FIG. 10C is a plot showing selected peptides inhibit rEfb-O binding to immobilized Fg. Increasing concentrations of Efb peptides were incubated with 5 nM rEfb-O in Fg-coated microtiter wells. To identify the residues in Efb-O that are important for Fg binding an Alanine scanning approach was used. A panel of Efb-O variant peptides were synthesized where each residues in the sequence is individually replaced with Ala (or Ser when the native a.a. is Ala; FIG. 10A). The individual peptides are then examined for their ability to compete with the binding of rEfb-O (5 nM) to immobilized Fg. The inhibitory activity of the peptides was compared at a fixed concentration (2 μM) for each peptide (FIG. 10B) and at increasing concentrations for selected peptides (FIG. 10C). As the Efb-O sequence is found in a disordered segment of the protein, the peptides are likely to be very flexible in solution. Therefore it is reasonable to assume that a peptide's inhibitory activity reflects its relative affinity for Fg.

As expected, the control wild-type peptide sEfb-O efficiently blocked the corresponding recombinant protein rEfb-O from binding to Fg, demonstrating that peptide sEfb-O has full Fg binding activity compared to rEfb-O. Surprisingly Ala substitution of over 15 residues distributed throughout the 25 amino acid long Efb-O motif resulted in loss or significant reduction in inhibitory activity (FIG. 10B), suggesting that residues throughout the entire segment are involved in Fg-binding. The results revealed that peptides in which Ala replaces residues K¹, I³, H⁷, Y⁹, I¹¹, E¹³, F¹⁴, D¹⁶, G¹⁷, T¹⁸, F¹⁹, Y²¹, G²², R²⁴ and P²⁵ lose their ability to inhibit rEfb-O binding (shown in red color in FIG. 10B), indicating that these residues are critical for Efb-O to bind to Fg (FIG. 10B). Ala replacement of residues Ile³ and Glu¹³ resulting in peptides sEfb-O3 (I3A) and sEfb-O13 (E13A), respectively, showed a markedly reduced yet significant dose dependent inhibitory activity suggesting that the residues Ile³ and Glu¹³ play some but less important roles in the Fg interaction (FIG. 10C).

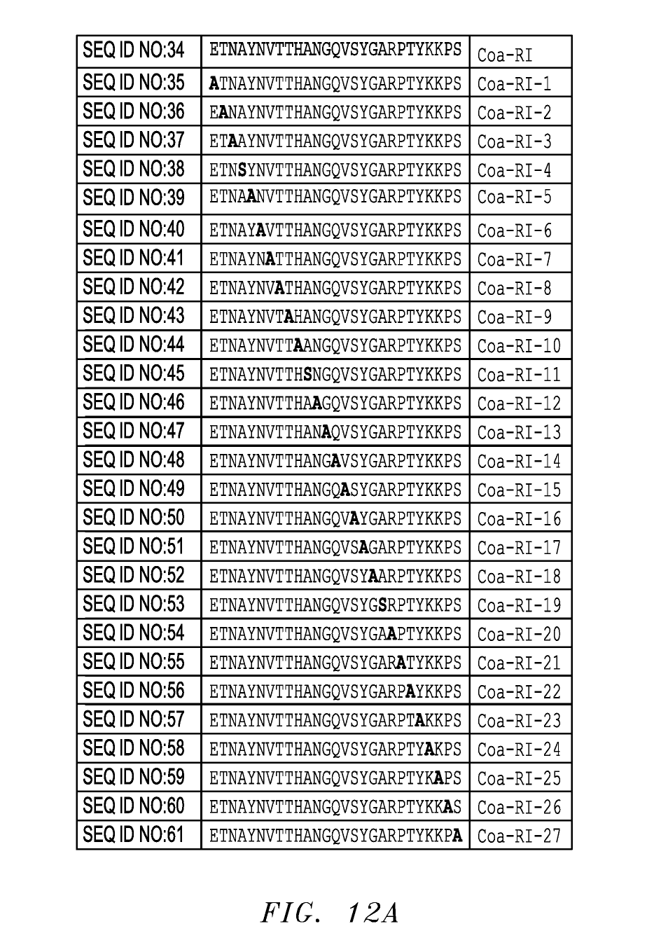

Coa-F contains an Efb like fibrinogen binding motif FIG. 11A is an image of a ClustalW alignment of amino acid sequence from Efb-O (Efb 68-98) and Coa from Newman strain (col-Newman). Sequence similarity was identified at Coa 474-505. Asterisks denote conserved residues and two dots represent similar residues. FIGS. 11B and 11C show a comparison of amino acid sequence of Efb-O with Coa 474-505 (FIG. 11B) and Coa 506-532 (FIG. 11C). Large letters in Efb-O indicate the residues important for Fg binding. The red letters show the identical residues and the yellow letters indicate the similar residues. FIGS. 11D and 11E shows the effect of Coa peptides on inhibition of rEfb-N(Efb 30-104) (FIG. 11D) and rCoa-C(Coa 311-636) (FIG. 11E) binding to Fg by the inhibition ELISA assays. Increasing concentration of Coa peptides was incubated with 2 nM GST fusion proteins in Fg-coated microtiter wells. Purple, sCoa-O; red, sCoa-RI; green, sEfb-O.

Next, sequences similar to the Fg-binding motifs in Efb were identified in Coa by comparing the amino acid sequence of Efb-O with Coa and found that a segment corresponding to residues Coa 474-505, named Coa-O, showed 56% amino acid identity and 75% similarity to that of the Efb-O sequence (FIG. 11A). Strikingly, of the residues in Efb-O determined to be important for Fg-binding (FIG. 10B) (letters in red) and FIG. 11B (large letters) all but three are conserved in Coa-O (FIG. 10B, shown in large red and orange letters), indicating that Coa-O likely constitutes an Efb-like Fg-binding motif. A peptide was synthesized that corresponds to the Coa-O sequence (sCoa-O) and determined its Fg binding activity in a competition ELISA. Microtiter wells were coated with Fg and binding of the recombinant N-terminal segment of Efb (rEfb-N), that harbors the two Fg binding sites, was quantitated in the presence of increasing concentration of different synthetic peptides. As expected the control peptide sEfb-O potently inhibited rEfb-N binding to the Fg surface (FIG. 11D). Peptide sCoa-O also acted as a potent inhibitor of the rEfb-N/Fg interaction (FIG. 11D), demonstrating that the Coa segment covered by residues 474-505 contains a Fg-binding site. The result also suggested that Coa-O likely competed with Efb-O for the same site in Fg.

It is noted that the repeated sequence of Coa contains remnants of the Efb Fibrinogen binding motif. The C-terminus of Coa harbors tandem repeats of a 27-residues segment and this region has been shown to bind Fg (FIGS. 9A and 9B). However, a Fg-binding motif has not been identified in the repeat region of Coa. An initial blast search failed to identify an Efb like Fg-binding motif in the Coa repeats but when the Efb-O sequence and the first repeat sequence were over-layered and showed that remnants of the Efb motif are also found in the Coa repeat sequences (FIG. 11C). Importantly the common residues are some of the ones shown to be critical for Efb-O binding to Fg (shown in large red letters). This observation suggests that the Coa repeats may bind Fg and possibly help define a functional register in the repeats. To investigate if the Coa repeats indeed have Fg binding activity, a peptide that constitutes the first 27 residues (Coa 506-532) (named sCoa-RI) was synthesized. This assumes that the functional Fg-binding repeats are directly following onto Coa-O (474-505). The Fg-binding activity of sCoa-RI was compared with those of sCoa-O and sEfb-O in competition ELISAs (FIG. 11D, 11E) where increasing concentrations of the peptides were used to inhibit the binding of rEfb-N(FIG. 11D) or rCoa-C (FIG. 11E) to Fg. All three peptides effectively inhibited rEfb-N binding to Fg, suggesting that the sCoa-RI also contains a Fg binding site likely targeting the same site in Fg as that recognized by Efb and Coa-O. Furthermore, sCoa-RI was a somewhat more effective inhibitor than sCoa-O despite the fact that the Coa-O sequence is more similar to that of Efb-O than Coa-RI. This observation suggests that some of the residues unique to Coa-RI are also participating in the Fg interaction. To determine what residues in Coa-RI are important for Fg-binding the Ala scanning approach was again used.

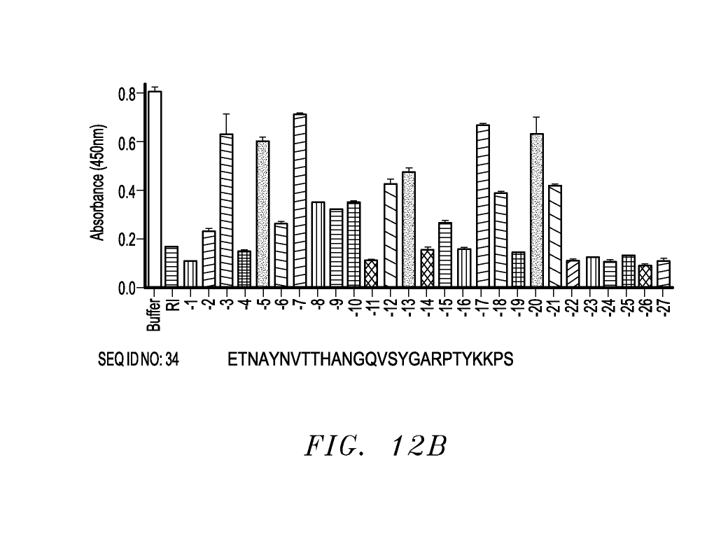

The residues in Coa-RI important for fibrinogen binding. FIG. 12A is a panel of coa-RI variant peptides were synthesized where each residue in the sequence is individually replaced with Ala (or Ser when the native a.a. is Ala). FIG. 12B is a sCoa-RI variant peptides (50 μM) inhibit GST-tagged rCoa-C(Coa 311-636) (2 nM) binding to immobilized Fg in solid phase assay. Wells were coated with 0.25 μg/well Fg. FIG. 12C is a comparison of amino acid sequence of Efb-O with Coa-RI. FIG. 12D is a Fg-binding register of tandem repeats in Coa. Asterisks denote the residues that are important for Fg binding. The peptide panel generated and tested is shown in FIG. 12A. Binding of a fixed concentration of rCoa-C (2 nM) to immobilized Fg was determined in the presence of a fixed concentration of these peptides (50 μM) (FIG. 12B). Interestingly, results revealed a similar pattern to that observed for Efb-O showing that the Ala substitution of over 13 residues distributed throughout the 27 amino acid long Coa-RI motif resulted in loss or significant reduction in inhibitory activity (FIG. 12B). This result suggests that, similar to Efb-O, residues in the entire segment of Coa-RI are involved in Fg binding. The results also showed that peptides in which alanine replaces residues N³, Y⁵, V⁷, T⁸, T⁹, H¹⁰, N¹², G¹³, V¹⁵ Y¹⁷G¹⁸ R²⁰ and P²¹ (FIG. 12B) lose their ability to inhibit rCoa-C binding (FIG. 12B, shown in red color), indicating that these residues are critical for Coa-RI to bind to Fg. Efb-O and Coa-RI sequences were compared to see how the critical residues in the two motifs line up. Strikingly, despite difference in numbers of residues and no extensive sequence identities between them, all but one the critical residues in Coa-RI correlate with similar residues in the corresponding position in Efb-O (FIG. 12C, marked with Asterisks). Furthermore, sequence comparisons within the different 27 residues repeats showed that the identified critical residues are conserved or replaced by similar residues (FIG. 12D).

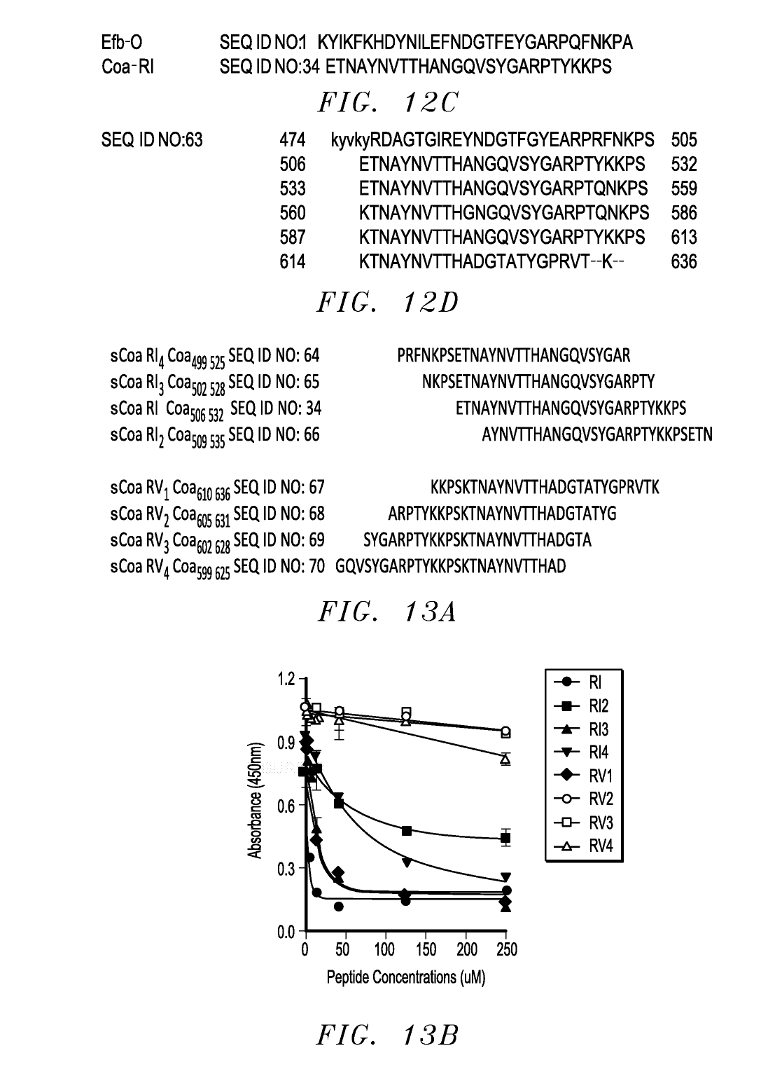

Identifying the functional register within the repeat region. FIG. 13A is a schematic presentation of Coa peptides. FIG. 13B is a plot of the effect of Coa peptides on inhibition of rCoa-C binding to fibrinogen. Increasing concentrations of synthetic peptides were incubated with 4 nM GST fusion protein in Fg-coated microtiter wells. Peptide sCoa-RI appears to be the most potent inhibitor. Red circle, sCoa-RI; orange square, coa-RI2 peptide; yellow triangle, coa-RI3; green inverted triangle, coa-RI4; green diamond, coa-RV1; blue circle, coa-RV2; black square, coa-RV3; red triangle, coa-RV4. In previous studies the repeated unit in Coa is proposed to start with residues alanine (A⁴⁹⁷) in S. aureus strain Newman. This register was based exclusively on sequence comparisons of Coa from different strains. To experimentally define a register of the repeats based on their Fg-binding function a panel of 27-residues peptides was synthesized and each peptide has 22-24 residues overlapped and largely covering the repeat I (RI) and repeat V (RV) (FIG. 13A). The Fg binding activities of these peptides were then investigated in a competition ELISA where the binding of rCoa-C to Fg coated microtiter wells were determined in the presence of increasing concentrations of peptide (FIG. 13B). It was observed that although peptides sCoa-RI, -RI₂, -RI₃, -RI₄ and -RV₁ showed some inhibitory activity, peptide sCoa-RI appears to be the most potent inhibitor among these eight peptides, suggesting that sCoa-RI (Coa 506-532) has the highest affinity for Fg (FIG. 13B) and that sCoa-RI likely represents a functional repeat unit that interacts with Fg. Notably, peptide sCoa-RV₂ (Coa 605-631), representing the previously proposed register, did not inhibit Fg binding in the experimental condition tested (FIG. 13B), indicating that this peptide has very low, if any, Fg binding activity. The results suggests that the functional (Fg binding) register of the repeat section is as outlined in FIG. 12D.

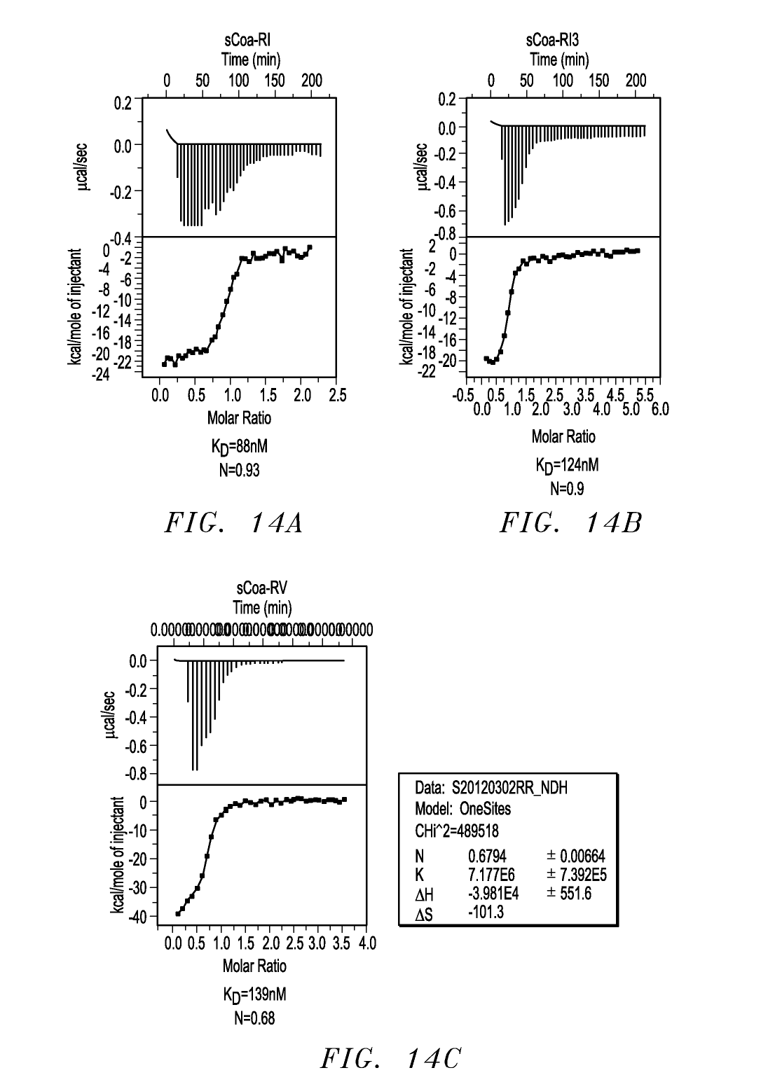

sCoa-RI, RI3 and RV1 bind to fibrinogen Coa-RI binds with higher affinity than other Coa peptides to Fg-D. FIGS. 14A-C shows a characterization of the interaction of Fg-D fragment with Coa peptides by VP-ITC. Binding isotherms for the interaction of Fg-D with Coa peptide sCoa-RI (FIG. 14A), sCoa-RI3 (FIG. 14B) and sCoa-RV1 (FIG. 14C) were generated by titrating the peptides (˜200 μM) into an ITC cell containing 10 μM Fg-D. The top panels show heat difference upon injection of coa peptides, and the low panels show integrated heat of injections. The data were fitted to a one-binding site model (bottom panels), and binding affinities are expressed as dissociation constants (K_{D}) or the reciprocal of the association constants determined by Microcal Origin software. N represents the binding ratio. To generate more quantitative binding data for the Coa peptide Fg interaction isothermal titration calorimetry and titrated the Coa peptides into a solution containing a fixed concentration of Fg-D fragments was used. Synthetic peptide sCoa-RI (Coa 506-532) bound to Fg-D fragment with a high affinity (K_{D}=88 nM) and a binding stoichiometry is 0.93 (FIG. 6A), suggesting that one molecule of sCoa-RI bound to one Fg-D molecule. Interactions between peptide sCoa-RI3 (Coa 502-528) and Fg-D fragments revealed an affinity of 124 nM (K_{D}); whereas sCoa-RV1 (Coa 610-636) had a K_{D} of 139 nM (FIG. 14B and FIG. 14C, respectively). These results corroborated with our competition ELISA results (FIG. 13B) and showed that sCoa-RI (Coa 506-532) bound Fg-D stronger than sCoa-RI3 (Coa 502-528) and sCoa-RV1 (Coa 610-636).

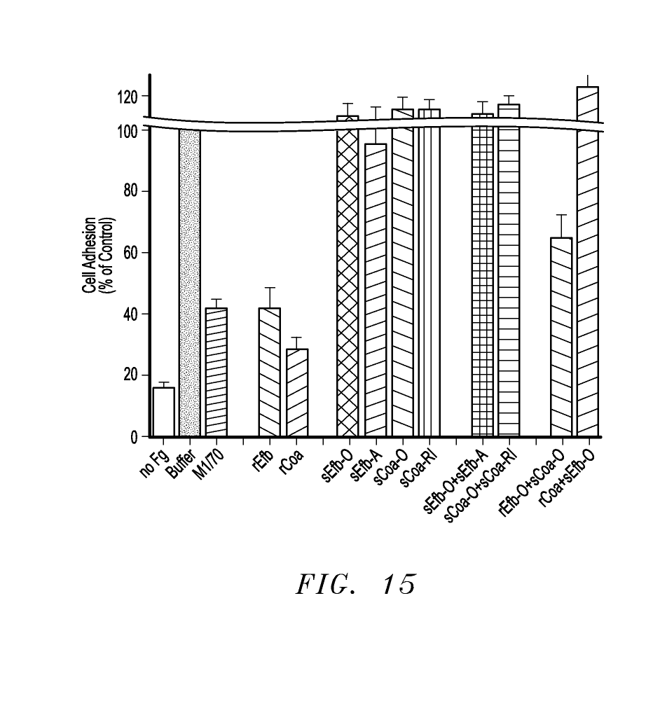

FIG. 15 shows Coa and Efb prevent monocytic cells from adherence to fibrinogen. Attachment of THP-1 cells to Fg immobilized on the 48-wells was inhibited by the addition of monoclonal αM antibody M1/70 (20 μg/ml), rEfb (0.2 μM) and rCoa (0.5 Addition of single peptide alone (sEfb-O, sEfb-A as well as sCoa-RI and sCoa-O, respectively, 0.5 μM each) or combination of two peptides together (sEfb-A+sEfb-O) or (sCoa-O+sCoa-RI), 0.5 μM each, did not inhibit THP-1 adherence. However, preincubation of sCoa-O peptide (50 μM) with rEfb (0.2 μM) or sEfb-O (50 μM) with rCoa (0.2 μM) reverses the inhibitory activities elicited by rEfb or rCoa. Error bars, S.D., n≧3. As Coa and Efb share similar Fg binding motif and could inhibit each other from binding to Fg, it was explored if Coa could also inhibit THP-1 monocytic cells adherence to Fg. THP-1 cells adhere to immobilized Fg primary through alphaMbeta2 integrin (also named Mac-1, CR3). In consistent to previously reported, antibody against alphaM (M1/70) inhibits THP-1 adherence to immobilized Fg (FIG. 15), confirming adherence of THP-1 cells to Fg is primary mediated by alphaMbeta2 integrin. Efb has been shown to block neutrophil-Fg interaction in an alphaMbeta2 dependent mechanism. Here as expected, Efb also efficiently inhibited THP-1 binding to Fg (FIG. 15). Similar to Efb, rCoa protein, that harbors multiple Fg binding motif, could also inhibit cell adherence to Fg surface. Interestingly, application of single individual synthetic peptides efb-O or efb-a that each contains one single Fg binding motif or in combination of two peptides (sEfb-O+sEfb-A) together did not show an effect. Similar phenomena were observed for sCoa-O and sCoa-RI, suggesting that inhibition of THP-1 cells adherence to Fg requires more than one Fg binding sites in one molecule. This is further supported by the observation that an excess amount of single peptide can partially, if not all, resolve the inhibitory effect mediated by rEfb or rCoa proteins (FIG. 15). In this situation, an excess amount of peptide sEfb-O or sCoa-O (50 μM) was mixed with rCoa (0.5 μM) or rEfb-N(0.2 μM), respectively, in the adherence assay. Coa is functionally related to Efb and that similar to Efb and Coa also inhibits monocytic-Fg interaction in alphaMbeta2 dependent process.

The pathogenic potential of S. aureus is a result of its multitude of virulence factors and their versatile interactions with multiple host factors. As a result S. aureus can survive and strive at many tissue sites in the host and cause a wide range of diseases. Fibrinogen is a surprisingly common target for many of the staphylococcal VF proteins. The known Fg-binding staphylococcal proteins largely fall into two groups: a family of structurally related cell-wall anchored proteins of the MSCRAMM type that include ClfA, ClfB, FnbpA, FnbpB and Bbp/SdrE) and a group of secreted smaller proteins (sometimes referred to as the SERAMs) that include Efb, Coa, von Willebrand factor-binding protein (vWbp), extracellular matrix binding protein (Emp) and extracellular adherence protein (Eap). The Fg-binding sites in the MSCRAMs are located to a segment of the proteins composed of two IgG-folded sub-domains that bind Fg by variants of the so called “dock, lock, and latch” mechanism. In this mechanism a short disordered segment of Fg docks in a trench formed between the two sub-domains through beta-complementation to a strand of the second sub-domain which subsequently triggers conformational changes in the MSCRAMM resulting in the subsequent steps.

The secreted proteins do not share a common domain organization and the mechanisms of Fg-binding used by these proteins remain largely unknown. However, these proteins do have some features in common One, they all interact with multiple ligands and Fg is the common ligand among them. Two, they all contribute to S. aureus abscess formation in animal infection models. Three, an intrinsically disordered region represents a significant part of each protein and it has previously been shown that the Fg binding sites in Efb is located to its disordered region. A disordered protein is particularly suited for accommodating multiple ligands since several interacting motifs can fit in a short segment of the protein and these motifs can be overlapping because the segment has structural plasticity. Furthermore amino acid sequence changes in a disordered protein segment are common since in these sections amino acid residue substitutions, deletions or additions can occur without interfering with a pre-existing structure. This tendency of sequence variations makes it particularly challenging to recognize interactive sequence motifs since these are often non-precise particularly if the motif is extended. The secreted staphylococcal Coa contains multiple copies of a Fg binding motif that functionally is similar to that previously identified in Efb's but that contains significant variations. Using an Alanine scanning approach the residues in the motifs critical for Fg binding were identified. Comparing these critical residues in the Efb and the Coa motifs we find that these are largely conserved and that the Coa and Efb motifs are variants of the same motif. This Fg-binding motif has several unique characteristics. Firstly, the motif consists of 25-27 residues long peptide. This is unusual long compared to other known and well characterized interactive motifs. Secondly, along the length of the motif almost every other residue is important for Fg binding but exchange for similar residues is tolerated.

The Efb/Coa Fg-binding motif has been searched out in other eukaryotic and prokaryotic proteins including other staphylococcal SERAMs but so far without any hits. vWbp is structurally and functionally similar to Coa in the way that vWbp also activates prothrombin through the N-terminal D1D2 domain of the protein in a non-proteolytic manner and subsequently converts soluble Fg to insoluble fibrin clots. vWbp also binds Fg and this binding site was initially located to the C-terminal putatively disordered region but a recent study located the Fg-binding activity to the D1D2 domain of vWbp. No significant parts of the Efb/Coa Fg-binding motif is seen in any part of vWbp.

Efb is capable of escaping phagocytosis by formation of Fg containing shield surrounding the bacteria surface. This shield may protect the bacteria from clearance since opsonizing antibodies and phagocytes will not access the bacteria. In Efb dependent shield Fg is brought to the surface of bacteria by Efb's ability to bind to microbial surface bound complement C3 through the C-terminal domains of the protein and recruits Fg through the N-terminal domain of the protein. Coa contains similar Efb's binding motif for Fg and therefore likely can form a Fg containing shield but Coa does not contain any known interaction with the bacterial surface. Therefor the Fg shield may not be formed on the bacterial surface but surrounding the colony as seen in an abscess. In fact Coa and Fg coincide in the core surrounding an abscess lesion and it is likely this core has a structural organization similar to the Fg protective shield formed by Efb. Also some of the Fg binding MSCRAMMs can assemble a protective Fg containing shield around staphylococcal cells, a mechanism that could explain the virulence potential of proteins like ClfA.

It is likely that the interaction of staphylococcal proteins with Fg induces a conformational change in the host molecule which may in turn increasing its tendency to aggregate. Efb binding to Fg results in a masking of the site in Fg recognized by the αMβ2/Mac-1 integrin. However, Efb effectively binds to a Fg variant where this site is mutated suggesting that this masking is not due to a direct competition for the site but possibly caused by a induced conformational change in Fg. Here experiments demonstrate that Coa harboring similar Fg binding motif can also inhibit THP-1 cell adherence through αMβ2/Mac-1 dependent mechanism suggesting that similar conformational changes can be induced by variants of the motif present in Efb and Coa. A more complete understanding of the molecular basis for the interaction of staphylococcal proteins interaction with Fg and the resulting Fg shield formed should lead to a better understanding of bacterial immune evasion strategies and may potential lead to novel strategies for the prevention and treatment of staphylococcal infections.

Secreted Fg binding proteins from S. aureus Coa and Efb are functionally related and locate Fg binding motifs to the intrinsically disordered section of the proteins. The residues in both the Efb and Coa Fg binding motifs were identified and it was conclude that these are preserved and span a surprisingly long segment of the protein. Also Coa contains multiples of this Fg-binding motif and define the functional register of the repeats in the disordered C-terminal region of Coa.

Bacterial Strains, Plasmids, and Culture Conditions-Escherichia coli XL-1 Blue was used as the host for plasmid cloning whereas E. coli BL21 or BL21(DE3)pLys were used for expression of GST- or His-tag fusion proteins. Chromosomal DNA from S. aureus strain Newman was used to amplify the Coagulase DNA sequence. E. coli XL-1 bule and BL21 containing plasmids were grown on LB media with ampicillin (100 μg/ml) and BL21(DE3)pLys containing plasmids were grown on LB media with ampicillin (100 μg/ml) and chloramphenicol (35 μg/ml).

Cloning of Coa construct-Chromosomal DNA from S. aureus strain Newman was used as template for all PCR reactions using the oligonucleotide primers described in supplement data. PCR products were digested with BamH I and Sal I and ligated into the pGEX-5x-1 vector or digested with BamHI and PstI and ligated into the pRSETA. Insertions were confirmed by DNA sequencing.

Expression and purification of recombinant Coa-Plasmids encoding N-terminal glutathione S-transferase (GST) or N-terminal 6× His-tagged Coa fusion proteins were expressed in either E. coli strain BL21 (GST tagged) or strain BL21(DE3)pLys (His-tagged). Bacteria were grown overnight at 37° C. in LB containing appropriate antibiotics as described above. The overnight cultures were diluted 1:20 into fresh LB medium and recombinant protein expression was induced with 0.2 mM IPTG for 2-3 hours. Bacteria were harvested by centrifugation and lysed using a French press. Soluble proteins were purified through glutathione-Sepharose-4B column or by Ni-chelating chromatography according to the manufacturer's manual. Purified proteins were dialysis into TBS and stored at −20° C. Protein concentrations were determined by the Bradford assay (Pierce). Recombinant Efb proteins were purified as previously described (12).

Enzyme-linked Immunosorbent Assay-96-well immulon 4HBX microtiter plates were coated with 0.25 lag/well full length human Fibrinogen (diluted in PBS, Enzyme research) overnight at 4° C. unless otherwise indicated. After blocking the wells with 3% BSA/PBS, recombinant Coa proteins were added and the plates were incubated for one hour. Bound Coa proteins were detected through incubation with horseradish peroxidase (HRP)-conjugated anti-His antibodies (10,000× dilution) or HRP-conjugated anti-GST polyclonal antibodies (5000× dilution) for one hour and quantified after adding the substrate 0-phenylenediamine dihydrochloride by measuring the resulting absorbance at 450 nm in an ELISA microplate reader.

In the case of peptide inhibition assay, various concentration of Efb or Coa peptides were mixed with fixed concentration of Coa-GST or Efb-GST fusion proteins (5-10 nM) in TBS and the bound GST fusion proteins were detected through incubation with HRP-conjugated rabbit anti-GST polyclonal antibodies (5000× dilution). All proteins were diluted in TBS containing 1% BSA and 0.05% Tween 20 and the ELISA assays were carried out at room temperature.

Isothermal titration calorimetry—The interaction between Coa peptides and the soluble, isolated D fragment of Fibrinogen was further characterized by isothermal titration calorimetry (ITC) using a VP-ITC microcalorimeter. The Fibrinogen-D fragment used in these studies was generated by digesting full length Fibrinogen with plasmin for 4 h and fractionating the digestion products by gel filtration chromatography. The ITC cell contained 10 μM Fibrinogen-D fragments and the syringe contained 150-200 μM Coa peptides in TBS (25 mM Tris, 3.0 mM KCl and 140 mM NaCl, pH 7.4). All proteins were filtered through 0.22 μm membranes and degassed for 20 minutes before use. The titrations were performed at 27° C. using a single preliminary injection of 2 μl of Coa peptide followed by 30˜40 injections of 5 μl with an injection speed of 0.5 μl s−1. Injections were spaced over 5 minute intervals at a stirring speed of 260 rpm. Raw titration data were fit to a one-site model of binding using MicroCal Origin version 5.0.

Cell adherence assay using cell lines-A monocytic cell line THP-1 cell stably expressing αMβ2 was maintained in RPMI1640 supplemented with 10% fetal bovine serum, 2 μM L-glutamine, 100 units/ml penicillin and 100 μg/ml streptomycin. Prior to use, cells were harvested by centrifuge, washed and suspended in RPMI 1640/1% human serum albumin. For cell adherence assays, 48-well plates were coated with 200 μl of Fibrinogen (10 μg/ml) overnight at 4° C. followed by 1 hour at 37° C. before blocking with 1% Polyvinylpyrrolidone (PVP 3600 kDa) for 45 minutes at 37° C. Subsequently, the cells were seeded 2×105/well in the presence or absence of Coa or Efb recombinant proteins or peptides and incubated at 37° C. for 25 minutes. Non-adherent cells were removed by washing gently three times with PBS/1% BSA. Adherent cells were quantitated with CyQuant kit according to the manufacturer's manual.

Bacterial strains, fluorescent labeling and supernatants. The present disclosure used the laboratory S. aureus strains Newman, SH1000, Reynolds and Wood 46 (with low expression of Protein A). The S. aureus strain KV27 and the S. epidermidis and E. coli strains were clinical isolates obtained within the UMCU. Targeted deletion (and complementation) of Efb in S. aureus Newman was described previously. All strains were cultured overnight on Tryptic Soy Blood Agar (BD) or Todd Hewitt Agar (with appropriate antibiotics) at 37° C. The capsule-expressing S. aureus strain Reynolds and its isogenic CP5-deficient mutant were a kind gift of Jean Lee (Harvard Medical School, Boston, USA). To optimize capsule expression, strain Reynolds was grown on Columbia Agar supplemented with 2% NaCl (CSA) for 24 hours at 37° C. For fluorescent labeling of strains, bacteria were resuspended in PBS and incubated with 0.5 mg/ml fluorescein isothiocyanate (FITC, Sigma) for 30 minutes on ice. Bacteria were washed twice with PBS, resuspended in RPMI medium with HSA and stored at −20° C. until further use. For in vivo experiments, S. aureus Newman and the Efb mutant were transformed with the pCM29 plasmid (kindly provided by Alexander Horswill, University of Iowa) allowing constitutive expression of the superfolder green fluorescent protein (sGFP) via the sarAP1 promoter. To isolate bacterial supernatants, WT and mutant strains were cultured overnight in Todd Hewitt Broth (THB) without antibiotics and subsequently sub cultured in fresh THB for 4 hours or 20 hours. Cultures were centrifuged at 13,000 rpm and collected supernatants were stored at −20° C. until further use.

Protein expression and purification. Recombinant Efb proteins were generated in E. coli as described previously. Briefly, (parts of) the efb gene from S. aureus strain Newman (without the signal peptide) were amplified by PCR and ligated into either the pGEX-5x-1 vector or the pRSETB vector for N-terminal fusions with glutathione S-transferase (GST) or polyhistidine respectively. Mutations of the Fg and C3 binding domains were introduced in pGEX plasmids containing full-length GST-Efb as described previously. Recombinant proteins were expressed and purified according to the manufacturer's manual. In all studies where wild-type Efb was compared with mutants, GST-tagged Efb were used. Otherwise His-tagged Efb was used.

ELISA. Microtiter plates were coated with human C3b or Fg, blocked with 3% BSA-PBS, and incubated with 6 nM Efb for one hour at room temperature. Efb binding was detected using peroxidase-conjugated rabbit anti-GST polyclonal antibodies and quantified using 0-phenylenediamine dihydrochloride. To study formation of C3b-Efb-Fg complexes, C3b-coated plates were incubated with Efb for one hour at room temperature. After washing, human Fg (50 nM) was added and detected through incubation with peroxidase-conjugated anti-Fg antibodies.

Preparation of Fg-D fragments. D fragments of Fg were generated by digestion of human Fg (Enzyme research) with plasmin (Enzyme research, 10 μg/15 mg Fg) in TB S containing 10 mM CaCl₂ for 4 hours at 37° C. as described earlier with modifications. D fragments (85 kD) were purified by gel filtration on Sephacryl S-200 and analyzed by SDS-PAGE.

Purification of human blood products. For preparation of plasma, venous blood from 10 healthy volunteers was collected in glass vacutainers (BD) containing the anticoagulant lepirudin (50 μg/ml). To prepare serum, blood was collected in glass vacutainers (BD) without anticoagulant and allowed to clot for 15 minutes at room temperature. Plasma and serum were collected after centrifugation for 10 minutes at 4000 rpm at 4° C., pooled and subsequently stored at −80° C. Complement-inactivated serum was prepared by incubation of serum for 30 min at 56° C. Human neutrophils were isolated freshly from heparinized blood using the Ficoll-Histopaque gradient method and used on the same day.

Mice. C57BL/6 female mice were purchased from Harlan-Winkelmann and used in studies when they were between 8 and 10 weeks of age. They were housed in microisolator cages and given food and water ad libitum.

Phagocytosis assays. Whole blood phagocytosis. FITC-labeled S. aureus KV27 (1×10⁸/ml) was incubated with freshly isolated human lepirudin blood (50%) and buffer or Efb (0.5 μM) in RPMI-0.05% HSA for 25 minutes at 37° C. The reaction was stopped using FACS lysing solution; samples were washed with RPMI-0.05% HSA and analyzed by flow cytometry using a FACSCalibur (BD). Gating of cells occurred on basis of forward and side scatter; for each sample the fluorescence intensity of 10,000 gated neutrophils was measured. Phagocytosis was expressed as the percentage of neutrophils that became fluorescent.

Phagocytosis with purified neutrophils and plasma/serum. FITC-labeled bacteria (5×10⁷/ml) were mixed with human serum or plasma for 2 minutes at 37° C. in the presence or absence of Efb. Freshly isolated neutrophils (5×10⁶/ml) were added and phagocytosis was allowed for 15 min at 37° C. The reaction was stopped by formaldehyde fixation and analyzed by flow cytometry. Alternatively, phagocytosis mixtures were cytospinned on glass slides and stained using Giemsa-based Diff-Quick solution. To analyze killing, phagocytosis mixtures were not fixed but incubated for an additional 90 minutes before they were diluted into ice-cold water (pH 11) and incubated for 15 minutes on ice to enable neutrophil lysis. Viable bacteria were quantified by colony enumeration. For Fg supplementation, 5% serum was supplemented with 50-200 μg/ml human or mouse Fg (kindly provided by Dr. Jay L. Degen; purified from plasma of wild type and Fgγ^{390-396A} mice). To analyze the influence of bacterial supernatants on phagocytosis, FITC-labeled S. aureus KV27 (2.5×10⁷ cfu) was pre-incubated with human serum for 30 min at 37° C. in Veronal Buffered Saline containing Ca²⁺ and Mg²⁺ (VBS⁺⁺). After washing in VBS⁺⁺-0.5% BSA, bacteria were incubated with (2-fold) diluted culture supernatants or purified Efb (250 nM) for 1 hour at 37° C. After washing, bacteria were incubated with purified Fg (60 μg/ml, Invitrogen) in RPMI-HSA for 1 hour at 37° C. and subsequently, neutrophils were added (7.5×10⁵ cells) and phagocytosis was allowed for 30 minutes at 37° C.

In vivo phagocytosis. S. aureus strain SH1000 was grown to mid-log phase, heat-inactivated for 60 minutes at 90° C., and fluorescently labeled with carboxyfluorescein. To induce infiltration of neutrophils within the peritoneal cavity, mice were intraperitoneally treated with 1 mg of carrageenan (Type IV1) 4 and 2 days prior to bacterial challenge. Subsequently, mice were intraperitoneally injected with 200 μl of a solution containing 10⁸ heat-inactivated carboxyfluorescein-labeled S. aureus SH1000 and Efb (1 μM). To compare WT and A Efb strains, mice were directly inoculated in the peritoneal cavity with 300 μl of GFP-expressing WT or A Efb S. aureus cultures grown to a late exponential phase. Mice were sacrificed 1 h thereafter, and their peritoneum was lavaged with sterile PBS. Lavage samples were centrifuged and pelleted cells were incubated with purified anti-CD32 antibodies to block the FcR, followed by PE-conjugated anti-mouse Gr-1 antibodies. Cells were washed and quenched with trypan blue (2 mg/ml). Samples were immediately subjected to flow-cytometric analysis using a FACScan. Neutrophils were gated according to their expression of Gr-1 antigen (FL2). Phagocytosis was expressed as the percentage of neutrophils that became fluorescent.

Alternative pathway hemolysis assay. Human serum (5%) was incubated with buffer or Efb proteins (1 μM) in HEPES-MgEGTA (20 mM HEPES, 5 mM MgCl2, 10 mM EGTA) for 15 min at RT. Rabbit erythrocytes were added and incubated for 60 min at 37° C. Mixtures were centrifuged and hemolysis was determined by measuring the absorbance of supernatants at 405 nm.

Immunoblotting. To analyze C3b deposition on the bacterial surface, S. aureus strain Wood46 (3×10⁸/ml) was incubated with 5% human plasma in the presence of Efb (0.5 μM), EDTA (5 mM) or buffer (HEPES; 20 nM HEPES, 5 mM CaCl₂, 2.5 mM MgCl₂, pH 7.4) for 30 min at 37° C. shaking at 1100 rpm. Bacteria were washed twice with PBS-0.1% BSA and boiled in Laemmli sample buffer containing Dithiothreitol. Samples were subjected to SDS-PAGE and subsequently transferred to a nitrocellulose membrane. C3b was detected using a peroxidase-labeled polyclonal anti-human C3 antibody and developed using Enhanced Chemiluminescence. To quantify Efb in bacterial supernatants, His-Efb and supernatants were run together on an SDS-PAGE gel. After transfer, blots were developed using a polyclonal sheep anti-Efb antibody, peroxidase-labeled donkey anti-sheep antibodies (Fluka Analytical) and ECL.

Flow cytometry assays with S. aureus. S. aureus strain Wood46 (3×10⁸/ml) was pre-incubated with human serum for 30 min at 37° C. in VBS⁺⁺ buffer, washed with VBS⁺⁺-0.5% BSA and incubated with Efb (0.5 μM) or 2-fold diluted culture supernatants for 1 hour at 37° C. shaking. After another washing step, bacteria were incubated with Alexa-488 conjugated Fg (60 μg/ml, Invitrogen) for 1 hour at 37° C. shaking. Washed bacteria were analyzed by flow cytometry using a FACSCalibur (BD). Bacteria were gated on the basis of forward and side scatter properties and fluorescence of 10,000 bacteria was analyzed. Alternatively, pre-opsonized bacteria were incubated with Efb (0.5 μM) and/or unlabeled Fg (200 μg/ml) for 1 hour at 37° C. shaking. Washed bacteria were incubated with soluble rCR1 (10 μg/ml), FITC-labeled F(ab′)₂ anti-human C3 antibody or anti-human IgG antibody for 30 min at 37° C. CR1 was detected using PE-labeled anti-CD35 antibodies; the IgG antibody was detected using goat-anti-mouse PE antibodies. Capsule expression on strain Reynolds was analyzed by incubating bacteria with polyclonal anti-CP5 rabbit serum and Phycoerythrin (PE)-conjugated goat anti-rabbit antibody.

Confocal microscopy. Samples were transferred to glass slides and air-dried. Membrane dye FM 5-95 was added and slides were covered with a coverslip. Confocal images were obtained using a Leica TCS SP5 inverted microscope equipped with a HCX PL APO 406/0.85 objective.

Transmission Electron Microscopy. S. aureus strain Wood 46 (3×10⁸) was incubated with human plasma (10%) in the presence or absence of Efb (0.5 μM) in HEPES⁺⁺ for 30 minutes at 37° C., washed once with PBS-1% BSA and adsorbed to 100 mesh hexagonal Formvar-carbon coated copper grids. Samples were contrasted with 0.4% uranyl acetate (pH 4.0) and 1.8% methylcellulose and analysed in a JEOL 1010 transmission electron microscope at 80 kV.

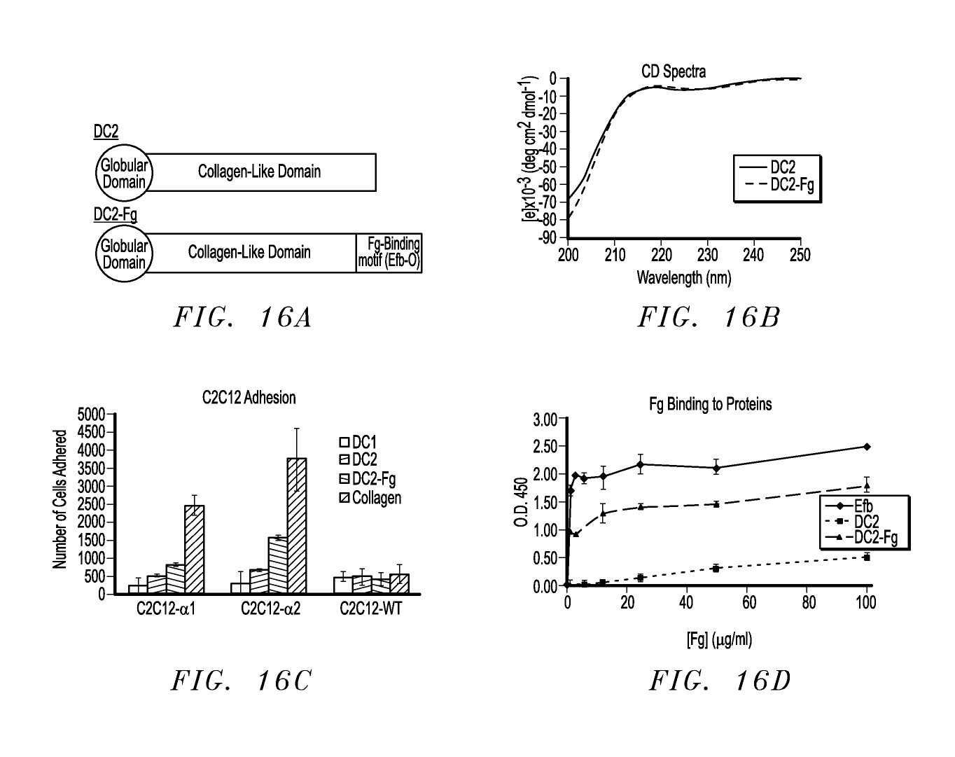

Recombinant proteins: The recombinant P163 protein was based upon the Scl2.28 sequence from S. pyogenes with the DNA codon optimized for E. coli expression. A hexahistidine tag was introduced at the N-terminus for use in purification. The GFPGER-containing variant described in Cosgriff-Hernandez, et al. and referred to as DC2 was utilized in these studies. The fibrinogen-binding DC2 variant (DC2-Fg) was generated using overlap extension polymerase chain reaction (PCR) with primers from Integrated DNA Technologies. The Fg binding motif Efb-O was inserted after position 301 Gln in DC2 shown in FIG. 16A. FIG. 16A is a Schematic representation of DC2-Fg with fibrinogen (Fg) binding motif Efb-O. The inserted Efb-O amino acid sequence is SEQ ID NO: 1 KYIKFKHDYN ILEFNDGTFE YGARPQFNKP A. The insertion was verified by sequencing (GENEWIZ, South Plainfield, N.J.).

Recombinant proteins were expressed in E. coli BL21 (Novagen). Purification was carried out by affinity chromatography on a StrepTrap HP column and subsequent dialysis against 20 mM acetic acid (regenerated cellulose, MWCO=12-14 kDa). Protein purity was assessed by sodium dodecyl sulfate polyacrylamide gel electrophoresis (SDS-PAGE) followed by Coomassie Blue staining. Protein concentrations were measured using the DC protein assay. Circular dichroism (CD) was utilized to confirm triple helix retention with the insertion as previously described.

Integrin interactions with DC2-Fg: All cell culture supplies were purchased from Life Technologies and used as received unless otherwise noted. To assess retention of integrin binding in DC2-Fg, adhesion of (i) C2C12 cells, which do not natively express integrin α1 or α2 subunits, (ii) C2C12 cells modified to stably express human integrin al subunits (C2C12-α1), and (iii) C2C12 cells modified to stably express human integrin α2 subunits (C2C12-α2) was measured. Mouse myoblast C2C12, C2C12-α1, and C2C12-α2 cells were cultured in Dulbecco's modified Eagle's medium (DMEM) with 10 vol % fetal bovine serum (FBS) and 1 vol % penicillin-streptomycin, 1 mg ml⁻¹ geneticin, or 10 μg ml⁻¹ puromycin, respectively. To assess C2C12 cell adhesion, 48 well tissue culture polystyrene (TCPS) plates were coated with 10 μg of DC1 (negative control—no integrin binding sites), DC2, DC2-Fg, or collagen type I (positive control) overnight at 4° C. Proteins were coated in triplicate for each cell type. Wells were blocked with 4 wt % bovine serum albumin (BSA) in PBS for 1 hour at room temperature and rinsed with sterile PBS. Cells were adapted to serum-free media (DMEM with 1 mM CaCl₂, 1 mM MgCl₂, and appropriate antibiotic) for 12 hr prior to trypsanization and seeding at 5,000 cells cm⁻¹. After 1 hour, cells were washed three times with warm PBS and lysed with 1% Triton-X 100 for 30 minutes at 37° C. Lysates from samples and from known standards were transferred to a 96 well plate, and cell numbers were measured with the CYTOTOX 96® NON-RADIOACTIVE CYTOTOXICITY ASSAY. Briefly, 50 μl of samples were incubated with 50 μl of substrate solution for 30 min at room temperature. Then, 50 μl of stop solution was added to each well, and the absorbance was read at 490 nm. Cell numbers were quantified using standards of known cell numbers for each cell line.

Solid phase binding assays: Microtiter wells were coated with 1 μg of DC2, DC2-Fg, or Efb overnight at 4° C. to assess fibrinogen adhesion to DC proteins. Coated wells were blocked with 4 wt % BSA in PBS for 1 hr at room temperature. Fibrinogen was added to each protein-coated well in a serial dilution from 100 to 0 μg/well (0.3 to 0 μM). After 1 hour of incubation at room temperature, a sheep anti-fibrinogen antibody was applied to the wells (1:1000 dilution) for 1 hour at room temperature. A HRP-labelled secondary antibody to sheep was applied to the wells for 1 hour at room temperature, and SigmaFast OPD was utilized to detect bound fibrinogen via an absorbance reading at 450 nm on a Thermomax plate reader. Studies were performed in triplicate, and plates were washed three times between each step with 200 μl of PBS with 0.1 vol % Tween-20.

FIG. 16B is an image of a circular dichroism (CD) spectra of DC2 and DC2-Fg. Peak at 220 nm is indicative of triple helix. DC2-Fg was successfully expressed and purified. The CD spectrum of DC2-Fg indicates that the protein retains the triple helical conformation of DC2 with the insertion, as demonstrated by the positive peak at 220 nm. FIG. 16C is plot of the integrin α1 and α2 subunit expressing C2C12 cell adhesion to DC1 (no integrin binding site), DC2 (binding site for integrins α1 and α2), DC2-FN (DC2 with fibrinogen binding site), and collagen (multiple binding sites for integrins α1 and α2). Retention of integrin binding with the Fg-binding insertion was assessed using C2C12 cells that express integrin α1 or α2 subunits. DC2 demonstrated an increase in C2C12-α1 and C2C12-α2 adhesion relative to DC1 (non-integrin binding negative control), as expected. The insertion of the Fg-binding motif, Efb-O, did not interfere with integrin binding, as demonstrated by C2C12-α1 and C2C12-α2 adhesion. In fact, DC2-Fg had significantly increased C2C12-α1 and C2C12-α2 adhesion relative to DC2 (p<0.05). This could be due to cell production of fibrinogen and subsequent binding to the Fg-binding motif in addition to interacting with the integrin-binding site in DC2-Fg. FIG. 16D is a graph showing fibrinogen binding to DC2, DC2-Fg, and Efb, as determined by solid phase binding assay. Fibrinogen interactions with DC2 and DC2-Fg were assessed using a solid phase binding assay. DC2 exhibited minimal to no fibrinogen binding, with no saturation in binding within the tested range of concentrations. Insertion of the Fg-binding motif, Efb-O, provided a large increase in fibrinogen binding, with an apparent K_{D} of ˜10 nM. This binding affinity approached that of Fg to Efb-O, with an apparent K_{D} of ˜1 nM. These results indicate that the Efb-based fibrinogen binding site, Efb-O, was successfully inserted into DC2 to provide a triple helical protein with controlled integrin binding and fibrinogen interactions. Statistical analyses were performed using GraphPad Prism 4.0 package and the differences between groups were analyzed for significance using the two-tailed Student's t-test.

It is contemplated that any embodiment discussed in this specification can be implemented with respect to any method, kit, reagent, or composition of the invention, and vice versa. Furthermore, compositions of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

1. A vaccine comprising:
a) a pharmacologically effective amount of a vaccine in a pharmaceutically acceptable excipient, comprising a modified extracellular fibrinogen binding protein comprising at least a portion of a modified N-terminus fibrinogen binding region, at least a portion of a modified C-terminus complement protein binding region, or both, wherein the modified extracellular fibrinogen binding protein results in inhibiting the fibrinogen binding, C3 binding, or both;
b) a pharmacologically effective amount of a vaccine in a pharmaceutically acceptable excipient, comprising a modified extracellular fibrinogen binding protein comprising at least a portion of a modified N-terminus fibrinogen binding region, at least a portion of a modified C-terminus complement protein binding region, or both, wherein the modified extracellular fibrinogen binding protein does not shield the surface-bound complement protein, an antibody or both from recognition by a phagocytic receptor; or
c) a pharmacologically effective amount of a vaccine in a pharmaceutically acceptable excipient, comprising a modified extracellular fibrinogen binding protein comprising at least a portion of a modified N-terminus fibrinogen binding region, at least a portion of a modified C-terminus complement protein binding region, or both, wherein the modified extracellular fibrinogen binding protein does not shield the staphylococcus bacterium from recognition by a phagocytic receptor.

2. A chimeric molecule of an extracellular fibrinogen binding protein (Efb) comprising:
a N-terminus fibrinogen binding region that binds a fibrinogen; and
a C-terminus complement protein binding region that binds a complement protein, wherein the chimeric molecule can modulate complement activity, modulate antibody binding, modulate recognition by a phagocytic receptor or a combination thereof.

3. A monoclonal and/or polyclonal antibody or antigen-binding fragment thereof that can specifically bind to a portion of a extracellular fibrinogen binding protein comprising:
a heavy and light chain variable regions that bind at least a portion of a N-terminus fibrinogen binding region of a extracellular fibrinogen binding protein, at least a portion of a C-terminus complement protein binding region of a extracellular fibrinogen binding protein, or both and results in the inhibition of fibrinogen binding, of complement protein binding, inhibition of the shielding of the staphylococcus bacterium from recognition by a phagocytic receptor or a combination thereof.

4. A pharmaceutical composition comprising:
a pharmacologically effective amount of a modified extracellular fibrinogen binding protein in a pharmaceutically acceptable excipient, wherein the modified extracellular fibrinogen binding protein comprises at least a portion of a N-terminus fibrinogen binding region, at least a portion of a C-terminus complement protein binding region, or both, wherein the modified extracellular fibrinogen binding protein results in inhibiting the fibrinogen binding, C3 binding, the surface-bound complement protein, an antibody or combination thereof.

5. A pharmaceutical composition comprising:
a monoclonal and/or polyclonal antibody or antigen-binding fragment thereof that can specifically bind to a portion of a extracellular fibrinogen binding protein comprising
a heavy and light chain variable regions that bind at least a portion of a N-terminus fibrinogen binding region of a extracellular fibrinogen binding protein, at least a portion of a C-terminus complement protein binding region of a extracellular fibrinogen binding protein, or both and results in the inhibition of fibrinogen binding, of complement protein binding, inhibition of the shielding of the staphylococcus bacterium from recognition by a phagocytic receptor or a combination thereof.

6. A pharmaceutical composition for use in the treatment of an infection comprising:
a) a pharmacologically effective amount of a modified extracellular fibrinogen binding protein in a pharmaceutically acceptable excipient, wherein the modified extracellular fibrinogen binding protein comprises at least a portion of a N-terminus fibrinogen binding region, at least a portion of a C-terminus complement protein binding region, or both, wherein the modified extracellular fibrinogen binding protein results in inhibiting the fibrinogen binding, C3 binding, the surface-bound complement protein, an antibody or combination thereof; or
b) a pharmacologically effective amount of a monoclonal and/or polyclonal antibody or antigen-binding fragment thereof that can specifically bind to a portion of a extracellular fibrinogen binding protein comprising a heavy and light chain variable regions that bind at least a portion of a N-terminus fibrinogen binding region of a extracellular fibrinogen binding protein, at least a portion of a C-terminus complement protein binding region of a extracellular fibrinogen binding protein, or both and results in the inhibition of fibrinogen binding, of complement protein binding, inhibition of the shielding of the staphylococcus bacterium from recognition by a phagocytic receptor or a combination thereof.

7. The composition of claim 1, wherein the at least a portion of a N-terminus fibrinogen binding region is selected from SEQ. ID NO: 3-61, preferably SEQ. ID NO: 3-30 or SEQ. ID NO: 35-61.

8. The composition of claim 1, wherein the at least a portion of a N-terminus fibrinogen binding region is selected from SEQ. ID NO: 3, 4, 5, 6, 7, 8, 9, 10, 11, 12, 13, 14, 15, 16, 17, 18, 19, 20, 21, 22, 23, 24, 25, 26, 27, 28, 29, 30, 35, 36, 37, 38, 39, 40, 41, 42, 43, 44, 45, 46, 47, 48, 49, 50, 51, 52, 53, 54, 55, 56, 57, 58, 59, 60, and 61.

9. The composition of claim 1, wherein the fibrinogen binding protein is Efb, Coa or both.

10. The composition of claim 1, further comprising an antigen selected from SpA, SpA variant, Emp, EsxA, EsxB, EsaC, Eap, EsaB, Coa, vWbp, vWh, Hla, SdrC, SdrD, SdrE, IsdA, IsdB, IsdC, ClfA, ClfB, SasF Sta006, Sta011, Hla and EsxA-EsxB.

11. A pharmaceutical composition for the targeted delivery of an active agent comprising:
a pharmacologically effective amount of a modified extracellular fibrinogen binding protein connected to a collagen-like domain, a globular domain or both and disposed in a pharmaceutically acceptable carrier, wherein the modified extracellular fibrinogen binding protein comprises a N-terminus fibrinogen binding region that binds a fibrinogen delivering the collagen-like domain, a globular domain or both to the fibrinogen.

12. The composition of claim 11, wherein the at least a portion of a N-terminus fibrinogen binding region is SEQ. ID NO: 2 or SEQ. ID NO: 34.

13. The composition of claim 11, wherein the collagen-like domain, a globular domain or both form a hydrogel.

14. The composition of claim 11, further comprising an antigen selected from SpA, SpA variant, Emp, EsxA, EsxB, EsaC, Eap, EsaB, Coa, vWbp, vWh, Hla, SdrC, SdrD, SdrE, IsdA, IsdB, IsdC, ClfA, ClfB, SasF Sta006, Sta011, Hla and EsxA-EsxB.

15. A method for making a monoclonal antibody comprising the steps of:
providing an effective amount of a composition comprising a modified extracellular fibrinogen binding protein having a N-terminus modified fibrinogen binding protein that does not bind fibrinogen, a C-terminus modified complement binding protein that does not bind a complement protein or both;
producing an antibody pool of the modified extracellular fibrinogen binding protein, the C-terminus modified complement binding protein, or both;
screening the antibody pool to detect active antibodies; wherein the active antibodies inhibit the fibrinogen binding to extracellular fibrinogen binding protein;
separating the active antibodies; and
adding the active antibodies to a pharmaceutically acceptable carrier.

16. A method for making a vaccine comprising the steps of:
providing an effective amount of a composition comprising a modified extracellular fibrinogen binding protein having a N-terminus modified fibrinogen binding protein that does not bind fibrinogen, a C-terminus modified complement binding protein that does not bind a complement protein or both and further comprising an antigen selected from SpA, SpA variant, Emp, EsxA, EsxB, EsaC, Eap, EsaB, Coa, vWbp, vWh, Hla, SdrC, SdrD, SdrE, IsdA, IsdB, IsdC, ClfA, ClfB, SasF Sta006, Sta011, Hla and EsxA-EsxB.

17. The method of claim 15, wherein the N-terminus modified fibrinogen binding protein has 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9, or 99.99% homology to SEQ ID NO: 2; SEQ ID NO: 34; or both.

18. The method of claim 15, wherein the at least a portion of a N-terminus fibrinogen binding region is selected from SEQ. ID NO: 3-30; from SEQ. ID NO: 35-61; or both.

19. The method of claim 15, wherein the at least a portion of a N-terminus modified fibrinogen binding protein is selected from SEQ. ID NO: 3, 4, 5, 6, 7, 8, 9, 10, 11, 12, 13, 14, 15, 16, 17, 18, 19, 20, 21, 22, 23, 24, 25, 26, 27, 28, 29, and 30 or from SEQ. ID NO: 35, 36, 37, 38, 39, 40, 41, 42, 43, 44, 45, 46, 47, 48, 49, 50, 51, 52, 53, 54, 55, 56, 57, 58, 59, 60, 61.


SEQUENCE LISTING (70 sequences)

SEQ ID NO: 1 - protein, 31 aa, Artificial Sequence
  Synthetic peptide
KYIKFKHDYNILEFNDGTFEYGARPQFNKPA

SEQ ID NO: 2 - protein, 28 aa, Artificial Sequence
  Synthetic peptide
KYIKFKHDYNILEFNDGTFEYGARPQFN

SEQ ID NO: 3 - protein, 28 aa, Artificial Sequence
  Synthetic peptide
AYIKFKHDYNILEFNDGTFEYGARPQFN

SEQ ID NO: 4 - protein, 28 aa, Artificial Sequence
  Synthetic peptide
KAIKFKHDYNILEFNDGTFEYGARPQFN

SEQ ID NO: 5 - protein, 28 aa, Artificial Sequence
  Synthetic peptide
KYAKFKHDYNILEFNDGTFEYGARPQFN

SEQ ID NO: 6 - protein, 28 aa, Artificial Sequence
  Synthetic peptide
KYIAFKHDYNILEFNDGTFEYGARPQFN

SEQ ID NO: 7 - protein, 28 aa, Artificial Sequence
  Synthetic peptide
KYIKAKHDYNILEFNDGTFEYGARPQFN

SEQ ID NO: 8 - protein, 28 aa, Artificial Sequence
  Synthetic peptide
KYIKFAHDYNILEFNDGTFEYGARPQFN

SEQ ID NO: 9 - protein, 28 aa, Artificial Sequence
  Synthetic peptide
KYIKFKADYNILEFNDGTFEYGARPQFN

SEQ ID NO: 10 - protein, 28 aa, Artificial Sequence
  Synthetic peptide
KYIKFKHAYNILEFNDGTFEYGARPQFN

SEQ ID NO: 11 - protein, 28 aa, Artificial Sequence
  Synthetic peptide
KYIKFKHDANILEFNDGTFEYGARPQFN

SEQ ID NO: 12 - protein, 28 aa, Artificial Sequence
  Synthetic peptide
KYIKFKHDYAILEFNDGTFEYGARPQFN

SEQ ID NO: 13 - protein, 28 aa, Artificial Sequence
  Synthetic peptide
KYIKFKHDYNALEFNDGTFEYGARPQFN

SEQ ID NO: 14 - protein, 28 aa, Artificial Sequence
  Synthetic peptide
KYIKFKHDYNIAEFNDGTFEYGARPQFN

SEQ ID NO: 15 - protein, 28 aa, Artificial Sequence
  Synthetic peptide
KYIKFKHDYNILAFNDGTFEYGARPQFN

SEQ ID NO: 16 - protein, 28 aa, Artificial Sequence
  Synthetic peptide
KYIKFKHDYNILEANDGTFEYGARPQFN

SEQ ID NO: 17 - protein, 28 aa, Artificial Sequence
  Synthetic peptide
KYIKFKHDYNILEFADGTFEYGARPQFN

SEQ ID NO: 18 - protein, 28 aa, Artificial Sequence
  Synthetic peptide
KYIKFKHDYNILEFNAGTFEYGARPQFN

SEQ ID NO: 19 - protein, 28 aa, Artificial Sequence
  Synthetic peptide
KYIKFKHDYNILEFNDATFEYGARPQFN

SEQ ID NO: 20 - protein, 28 aa, Artificial Sequence
  Synthetic peptide
KYIKFKHDYNILEFNDGAFEYGARPQFN

SEQ ID NO: 21 - protein, 28 aa, Artificial Sequence
  Synthetic peptide
KYIKFKHDYNILEFNDGTAEYGARPQFN

SEQ ID NO: 22 - protein, 28 aa, Artificial Sequence
  Synthetic peptide
KYIKFKHDYNILEFNDGTFAYGARPQFN

SEQ ID NO: 23 - protein, 28 aa, Artificial Sequence
  Synthetic peptide
KYIKFKHDYNILEFNDGTFEAGARPQFN

SEQ ID NO: 24 - protein, 28 aa, Artificial Sequence
  Synthetic peptide
KYIKFKHDYNILEFNDGTFEYAARPQFN

SEQ ID NO: 25 - protein, 28 aa, Artificial Sequence
  Synthetic peptide
KYIKFKHDYNILEFNDGTFEYGSRPQFN

SEQ ID NO: 26 - protein, 28 aa, Artificial Sequence
  Synthetic peptide
KYIKFKHDYNILEFNDGTFEYGAAPQFN

SEQ ID NO: 27 - protein, 28 aa, Artificial Sequence
  Synthetic peptide
KYIKFKHDYNILEFNDGTFEYGARAQFN

SEQ ID NO: 28 - protein, 28 aa, Artificial Sequence
  Synthetic peptide
KYIKFKHDYNILEFNDGTFEYGARPAFN

SEQ ID NO: 29 - protein, 28 aa, Artificial Sequence
  Synthetic peptide
KYIKFKHDYNILEFNDGTFEYGARPQAN

SEQ ID NO: 30 - protein, 28 aa, Artificial Sequence
  Synthetic peptide
KYIKFKHDYNILEFNDGTFEYGARPQFA

SEQ ID NO: 31 - protein, 121 aa, Artificial Sequence
  Synthetic peptide
EGSSSKLEIKPQGTESTLKGTQGESSDIEVKPQATETTEASQYGPRPQFNKTPKYVKYRDAGTGIREYNDGTFGYEARPRFNKPSETNAYNVTTHANKGQVSYGARPTYKKPSETNAYNVT

SEQ ID NO: 32 - protein, 32 aa, Artificial Sequence
  Synthetic peptide
KYVKYRDAGTGIREYNDGTFGYEARPRFNKPS

SEQ ID NO: 33 - protein, 26 aa, Artificial Sequence
  Synthetic peptide
ETNAYNVTHANGQVSYGARPTYKKPS

SEQ ID NO: 34 - protein, 27 aa, Artificial Sequence
  Synthetic peptide
ETNAYNVTTHANGQVSYGARPTYKKPS

SEQ ID NO: 35 - protein, 27 aa, Artificial Sequence
  Synthetic peptide
ATNAYNVTTHANGQVSYGARPTYKKPS

SEQ ID NO: 36 - protein, 27 aa, Artificial Sequence
  Synthetic peptide
EANAYNVTTHANGQVSYGARPTYKKPS

SEQ ID NO: 37 - protein, 27 aa, Artificial Sequence
  Synthetic peptide
ETAAYNVTTHANGQVSYGARPTYKKPS

SEQ ID NO: 38 - protein, 27 aa, Artificial Sequence
  Synthetic peptide
ETNSYNVTTHANGQVSYGARPTYKKPS

SEQ ID NO: 39 - protein, 27 aa, Artificial Sequence
  Synthetic peptide
ETNAANVTTHANGQVSYGARPTYKKPS

SEQ ID NO: 40 - protein, 27 aa, Artificial Sequence
  Synthetic peptide
ETNAYAVTTHANGQVSYGARPTYKKPS

SEQ ID NO: 41 - protein, 27 aa, Artificial Sequence
  Synthetic peptide
ETNAYNATTHANGQVSYGARPTYKKPS

SEQ ID NO: 42 - protein, 27 aa, Artificial Sequence
  Synthetic peptide
ETNAYNVATHANGQVSYGARPTYKKPS

SEQ ID NO: 43 - protein, 27 aa, Artificial Sequence
  Synthetic peptide
ETNAYNVTAHANGQVSYGARPTYKKPS

SEQ ID NO: 44 - protein, 27 aa, Artificial Sequence
  Synthetic peptide
ETNAYNVTTAANGQVSYGARPTYKKPS

SEQ ID NO: 45 - protein, 27 aa, Artificial Sequence
  Synthetic peptide
ETNAYNVTTHSNGQVSYGARPTYKKPS

SEQ ID NO: 46 - protein, 27 aa, Artificial Sequence
  Synthetic peptide
ETNAYNVTTHAAGQVSYGARPTYKKPS

SEQ ID NO: 47 - protein, 27 aa, Artificial Sequence
  Synthetic peptide
ETNAYNVTTHANAQVSYGARPTYKKPS

SEQ ID NO: 48 - protein, 27 aa, Artificial Sequence
  Synthetic peptide
ETNAYNVTTHANGAVSYGARPTYKKPS

SEQ ID NO: 49 - protein, 27 aa, Artificial Sequence
  Synthetic peptide
ETNAYNVTTHANGQASYGARPTYKKPS

SEQ ID NO: 50 - protein, 27 aa, Artificial Sequence
  Synthetic peptide
ETNAYNVTTHANGQVAYGARPTYKKPS

SEQ ID NO: 51 - protein, 27 aa, Artificial Sequence
  Synthetic peptide
ETNAYNVTTHANGQVSAGARPTYKKPS

SEQ ID NO: 52 - protein, 27 aa, Artificial Sequence
  Synthetic peptide
ETNAYNVTTHANGQVSYAARPTYKKPS

SEQ ID NO: 53 - protein, 27 aa, Artificial Sequence
  Synthetic peptide
ETNAYNVTTHANGQVSYGSRPTYKKPS

SEQ ID NO: 54 - protein, 27 aa, Artificial Sequence
  Synthetic peptide
ETNAYNVTTHANGQVSYGAAPTYKKPS

SEQ ID NO: 55 - protein, 27 aa, Artificial Sequence
  Synthetic peptide
ETNAYNVTTHANGQVSYGARATYKKPS

SEQ ID NO: 56 - protein, 27 aa, Artificial Sequence
  Synthetic peptide
ETNAYNVTTHANGQVSYGARPAYKKPS

SEQ ID NO: 57 - protein, 27 aa, Artificial Sequence
  Synthetic peptide
ETNAYNVTTHANGQVSYGARPTAKKPS

SEQ ID NO: 58 - protein, 27 aa, Artificial Sequence
  Synthetic peptide
ETNAYNVTTHANGQVSYGARPTYAKPS

SEQ ID NO: 59 - protein, 27 aa, Artificial Sequence
  Synthetic peptide
ETNAYNVTTHANGQVSYGARPTYKAPS

SEQ ID NO: 60 - protein, 27 aa, Artificial Sequence
  Synthetic peptide
ETNAYNVTTHANGQVSYGARPTYKKAS

SEQ ID NO: 61 - protein, 27 aa, Artificial Sequence
  Synthetic peptide
ETNAYNVTTHANGQVSYGARPTYKKPA

SEQ ID NO: 62 - skipped

SEQ ID NO: 63 - protein, 163 aa, Artificial Sequence
  Synthetic peptide
KYVKYRDAGTGIREYNDGTFGYEARPRFNKPSETNAYNVTTHANGQVSYGARPTYKKPSETNAYNVTTHANGQVSYGARPTQNKPSKTNAYNVTTHGNGQVSYGARPTQNKPSKTNAYNVTTHANGQVSYGARPTYKKPSKTNAYNVTTHADGTATYGPRVTK

SEQ ID NO: 64 - protein, 27 aa, Artificial Sequence
  Synthetic peptide
PRFNKPSETNAYNVTTHANGQVSYGAR

SEQ ID NO: 65 - protein, 27 aa, Artificial Sequence
  Synthetic peptide
NKPSETNAYNVTTHANGQVSYGARPTY

SEQ ID NO: 66 - protein, 27 aa, Artificial Sequence
  Synthetic peptide
AYNVTTHANGQVSYGARPTYKKPSETN

SEQ ID NO: 67 - protein, 27 aa, Artificial Sequence
  Synthetic peptide
KKPSKTNAYNVTTHADGTATYGPRVTK

SEQ ID NO: 68 - protein, 27 aa, Artificial Sequence
  Synthetic peptide
ARPTYKKPSKTNAYNVTTHADGTATYG

SEQ ID NO: 69 - protein, 27 aa, Artificial Sequence
  Synthetic peptide
SYGARPTYKKPSKTNAYNVTTHADGTA

SEQ ID NO: 70 - protein, 27 aa, Artificial Sequence
  Synthetic peptide
GQVSYGARPTYKKPSKTNAYNVTTHAD